(19) United States
(12) Patent Application Publication
(10) Pub. No.: US 2025/0281626 A1
(43) Pub. Date: Sep. 11, 2025

(54) MANNOSE 3 GLYCAN-MEDIATED PROTEIN DEGRADATION

(71) Applicant: GlycoEra AG, Wädenswil (CH)

(72) Inventors: Amirreza Faridmoayer, Oberengstringen (CH); Rainer Follador, Zurich (CH); Manuela Mally, Watt (CH); Jonathan Albert BACK, Villers-le-Lac (FR)

(21) Appl. No.: 18/283,725

(22) PCT Filed: Mar. 22, 2022
(86) PCT No.: PCT/EP2022/057554

§ 371 Date: Sep. 22, 2023

Related U.S. Application Data
(60) Provisional application No. 63/164,991, filed on Mar. 23, 2021.

(51) Int. Cl. A61K 47/61; A61K 47/68

(52) U.S. Cl. CPC A61K 47/61; A61K 47/6835

(57) ABSTRACT

The present disclosure provides bifunctional binding proteins for mannose 3 glycan-mediated degradation. Such glycan modifications will improve current treatments and allow a better quality of life for patients. Accordingly, such bifunctional binding proteins are useful for treating and preventing various diseases.

### 1. CROSS-REFERENCE TO RELATED APPLICATIONS

This application is a U.S. National Stage Application of International Patent Application No. PCT/EP2022/057554, filed Mar. 22, 2022, which claims the benefit of U.S. Provisional Application No. 63/164,991, filed Mar. 23, 2021, each of which is incorporated by reference herein in its entirety.

### 2. FIELD

The present invention relates generally to bifunctional binding proteins having mannose 3 glycosylation and lysosomal targeting of same.

### 3. BACKGROUND

Glycan engagement with carbohydrate binding receptors enables different biological pathways. The glycan-receptor interaction is determined by the glycan structure. These essential biological pathways involved in: modulating immune responses, mediating protein clearance, protein turnover, and controlling trafficking of soluble glycoproteins, glycolipids and or any natural molecule containing a glycan moiety.

Endocytic lectins are involved in receptor-mediated endocytosis by capturing glycosylated proteins via specific glycan structures to mediate degradation. Endocytic lectins are ubiquitous in humans and can recognize various glycan structures. See, eg: Cummings, Richard D.; McEver, Rodger P. (Eds.) (2017): Essentials of Glycobiology [Internet]. 3rd edition: Cold Spring Harbor Laboratory Press [8]. Additional background on glycans and their receptors is provided in references [9] to [12].

Carbohydrate binding receptors are highly diverse and can be exploited by glycoengineering to develop novel therapeutics with unprecedented effectiveness for different diseases, including but not limited to: inflammatory, blood disorders, autoimmune and cancer. This allows development of novel therapeutics based on the concept of glycan-mediated protein degradation. Leveraging human glycan-mediated protein degradation through the site specific glycosylation of any proteins such as chemokine, cytokine, polypeptide or monoclonal antibodies can lead to novel therapeutics. To date, it has not been described to what degree Man3 containing glycans such as Man3GlcNAc2 assembled on a protein can efficiently bind to lectins recognizing mannose or receptors and mediate lysosomal degradation. The present invention shows a novel finding of a specific glycan mediated protein degradation.

The compositions and methods provided herein address the unmet medical need of patients suffering from various difficult to treat diseases such as cancer, autoimmune, and inflammatory diseases treated with the current standard of care,

### 4. SUMMARY OF THE INVENTION

In one aspect, provided herein is a bifunctional binding protein comprising a first moiety that specifically binds to a target protein and a second moiety comprising a glycan comprising the structure:

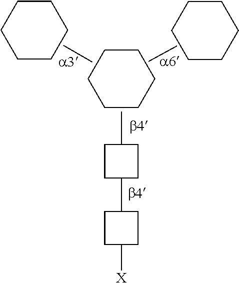

wherein the square represents an N-acetylglucosamine residue and the hexagon represents a mannose residue, and wherein X represents an amino acid residue of the bifunctional binding protein. In some embodiments, the N-glycan portion of a bifunctional binding protein consists of Mannotriose-di-(N-acetyl-D-glucosamine) (Man3GlcNAc2), ie, the mannose 3 glycan structure shown in this paragraph.

In certain embodiments, a bifunctional protein provided herein comprises a N-glycan with a mannose 3 structure as the terminal glycan. Specifically, any branched structure of the N-glycan on the GlcNAc2 part of the N-glycan can also be included. For example, the GlcNac that is directly linked to X can be fucosylated. As used in this application, the term “mannose 3 structure” or “Man3 structure” or “M3 structure” refers to an N-glycan with three terminal mannose residues.

In some embodiments, the target protein that the first moiety specifically binds comprises HER2, EGFR, HER3, VEGFR, CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, Frizzled receptors, Wnt, LRP5/6, CD38, CD73, TGF-β, Bombesin R, CAIX, CD13, CD44v6, Emmprin, Endoglin, EpCAM, EphA2, FAP-α, Folate R, GRP78, IGF-1R, Matriptase, Mesothelin, sMET/HGFR, MT1-MMP, MT6-MMP, PSCA, PSMA, Tn antigen, and uPAR, TSHRα, Myelin oligodendrocyte glycoprotein (MOG), AChR-α1, noncollagen domain 1 of the α3 chain of type IV collagen (α3NC1), ADAMTS13, Desmoglein-1/3, or GPIb/IX, GPIIb/IIIa, GPIa/IIa, NMDA receptor, glutamic acid decarboxylase (GAD), amphiphysin and gangliosides GM1, GD3, GQ1B, SIRPα, CCR2, CSF-1R, LILRB1, LILRB2, VEGF-R, CXCR4, CCL2, CXCL12, CSF-1 or CD47.

In some embodiments, the second moiety specifically binds to a mannose 3 receptor, a Cluster of Differentiation 206 (CD206) receptor, a DC-SIGN (Cluster of Differentiation 209 or CD209) receptor, a C-Type Lectin Domain Family 4 Member G (LSECTin) receptor, or a macrophage inducible Ca²⁺-dependent lectin receptor (Mincle). In some embodiments, the second moiety of the bifunctional binding protein specifically binds to any endocytic carbohydrate-binding receptor recognizing Man3GlcNAc2 structure. In some embodiments, the second moiety of the bifunctional binding protein specifically binds to langerin, a macrophage mannose 2 receptor, dectin-1, dectin-2, BDCA-2, DCIR, MBL, MDL, MICL, CLEC2, DNGR1, CLEC12B, DEC-205, asialoglycoprotein receptor (ASPGR), and mannose 6 phosphate receptor.

In some embodiments, second moiety comprises a glycan structure. In some embodiments, the glycan structure is mannose 3.

In some embodiments, the first moiety of the bifunctional binding protein comprises a heavy chain variable region or a light chain variable region. In some embodiments, the first moiety of the bifunctional binding protein comprises a Fab region of a monoclonal antibody.

In some embodiments, the bifunctional binding protein is an antibody. In some embodiments, the antibody is a monoclonal or polyclonal antibody. In some embodiments, the antibody is recombinant. In some embodiments, the antibody is humanized, chimeric or fully human. In some embodiments, the antibody has a glycan to protein ratio of 2 to 1, 4 to 1, 6 to 1, 8 to 1, or 10 to 1.

In some embodiments, the bifunctional binding protein is glycosylated at a predetermined and specific residue.

In some embodiments, the bifunctional binding protein is an autoantigen.

In some embodiments, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% of the glycans of the bifunctional binding protein have a mannose 3 glycan structure.

In some embodiments, the target protein is a cell surface molecule or a non-cell surface molecule. In some embodiments, the cell surface molecule is a receptor. In some embodiments, the non-cell surface molecule is an extracellular protein. In some embodiments, the extracellular protein is an autoantibody, a hormone, a cytokine, a chemokine, a blood protein, or a central nervous system (CNS) protein.

In some embodiments, the target protein is bound by the first moiety.

In one aspect, provided herein is a method of delivering a target protein to the endosome of a hepatic macrophage, such as a liver resident Kupffer cell (KCs) and/or liver sinusoidal endothelial cells (LSEC) and/or a monocyte-derived macrophage (MoMϕs), comprising: contacting the target protein with the bifunctional binding protein provided herein under conditions to mediate endocytosis of the target protein.

In one aspect, provided herein is a method of degrading a target protein comprising: contacting the target protein with the bifunctional binding protein provided herein under conditions to mediate lysosomal degradation of the target protein by a host cell.

In some embodiments, the target protein is upregulated in cancer or involved in cancer progression.

In some embodiments, the target protein upregulated in cancer or involved in cancer progression comprises HER2, EGFR, HER3, VEGFR CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2, Frizzled receptors, Wnt, LRP5/6, CSF-1R, SIRPα, CD38, CD73, LILRB2 or TGF-β.

In some embodiments, the target protein is an autoantibody of an autoimmune disease. In some embodiments, the target protein is an autoantigen in an autoimmune disease.

In some embodiments, the autoantibody in the autoimmune disease is an antibody binding to TSHRα, MOG (Myelin oligodendrocyte glycoprotein), AChR-α1, noncollagen domain 1 of the α3 chain of type IV collagen (α3NC1), ADAMTS13, Desmoglein-1/3, GPIb/IX, GPIIb/IIIa, GPIa/IIa, NMDA receptor, glutamic acid decarboxylase (GAD), amphiphysin, or gangliosides GM1, GD3 or GQ1B.

In some embodiments, the target protein is upregulated or expressed in a neurodegenerative disease. In some embodiments, the target protein upregulated or expressed in a neurodegenerative disease is alpha-synuclein, amyloid beta, complement cascade component, or build-up of aggregated misfolded light chains or aggregated misfolded transthyretin in systemic amyloidosis.

In some embodiments, the target protein is upregulated or expressed in tumor associated macrophages (TAMs). In some embodiments, the target protein is associated with TAMs recruitment in the tumor microenvironment. In other embodiments, the target protein is associated with TAMs depletion in the tumor microenvironment. In further embodiments, the target protein is associated with TAMs reprogramming in the tumor microenvironment.

In some embodiments, the target protein associated with TAMs comprises SIRPα, CCR2, CSF-1R, LILRB1, LILRB2, VEGF-R, or CXCR4. In other embodiments, the target protein associated with TAMs comprises CCL2, CXCL12, CSF-1 or CD47.

In some embodiments, the host cell is a myeloid cell, an immune cell, an endothelial cell, a parenchymal cell, or an epithelial cell. In some embodiments, the immune cell is a dendritic cell, a macrophage, a monocyte, a microglia cell, a granulocyte or a B lymphocyte.

In some embodiments, the host cell is any cell.

In some embodiments, the bifunctional binding protein enhances degradation of the target protein relative to degradation of the target protein in the presence of a bifunctional binding protein comprising a different second moiety.

In some embodiments, degradation is mediated by endocytosis or phagocytosis.

In one aspect, provided herein is a pharmaceutical composition comprising the bifunctional binding protein provided herein and a pharmaceutically acceptable carrier.

In one aspect, provided herein is a method of treating or preventing a disease in a patient comprising: administering to the patient the bifunctional binding protein provided herein, or the pharmaceutical composition provided herein.

In some embodiments, the disease is an autoimmune disease, a cancer or tumor, a liver disease, an inflammatory disorder, or a blood coagulation disorder. In some embodiments, the autoimmune disease is selected from Graves' Disease, Myasthenia Gravis, Anti-GBM Disease, Immune Thrombotic Thrombocytopenic Purpura, Acquired Pemphigus Vulgaris, Immune Thrombocytopenia, autoimmune encephalitis, Myelin oligodendrocyte glycoprotein antibody-associated disease (MOGAD), membranous nephropathy, systemic amyloidosis, and Guillain-Barré Syndrome.

In some embodiments, the administration step comprises intravenous injection, intraperitoneal injection, subcutaneous injection, transdermal injection, or intramuscular injection.

In one aspect, provided herein is a kit comprising the bifunctional binding protein provided herein, or the pharmaceutical composition provided herein and instructions for administering the bifunctional molecule or pharmaceutical composition to an individual in need thereof.

In some embodiments, the bifunctional binding protein or pharmaceutical composition is present in one or more unit dosages.

In some embodiments, the glycan further comprises a fucose residue at the N-acetylglusoamine that is directly attached to X. In some embodiments, X is an asparagine residue in the bifunctional binding protein.

In one aspect, provided herein is a bifunctional binding protein, wherein the bifunctional binding protein (i) specifically binds to a target protein and (ii) comprises an N-glycan of the structure:

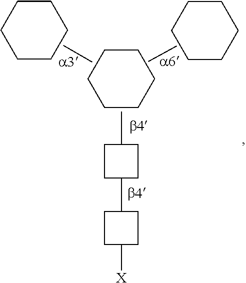

wherein the square represents an N-acetylglucosamine residue and the hexagon represents a mannose residue, and wherein X represents an amino acid residue of the bifunctional binding protein, wherein the N-glycan is linked to the bifunctional binding protein at 1, 2, 3, 4 or 5 N-glycosylation sites.

In some embodiments, the glycan further comprises a fucose residue at the N-acetylglusoamine that is directly attached to X. In some embodiments, X is an asparagine residue in the bifunctional binding protein.

In one aspect, provided herein is a population of bifunctional binding proteins, wherein for at least one of the N-glycosylation sites at a specified amino acid position of the bifunctional binding protein, at least 50%, at least 60%, at least 70%, at least 80%, at least 90% or 99% of N-glycosylation sites in the population are glycosylated with a mannose 3 N-glycan structure.

In some embodiments, the N-glycosylation site comprises one or more asparagine residues, wherein the asparagine residues are within a canonical consensus sequence N-X-S/T, N-X-C motifs (X can be any amino acids except proline), and non-canonical consensus motifs.

In some embodiments, the N-glycosylation site is introduced into the bifunctional protein by recombinant engineering.

In some embodiments, the recombinant engineering is performed adding an amino acid, deleting an amino acid, substituting an amino acid, or adding a glycotag. In some embodiments, the glycan consists a mannose 3 N-glycan structure.

In some embodiments, the target protein comprises HER2, EGFR, HER3, VEGFR, CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, Frizzled receptors, Wnt, LRP5/6, CD38, CD73, TGF-β, Bombesin R, CAIX, CD13, CD44, v6, CXCR4, ErbB-2, Her2, Emmprin, Endoglin, EpCAM, EphA2, FAP-α, Folate R, GRP78, IGF-1R, Matriptase, Mesothelin, sMET/HGFR, MT1-MMP, MT6-MMP, Muc-1, PSCA, PSMA, Tn antigen, and uPAR, TSHRα, AChR-α1, noncollagen domain 1 of the α3 chain of type IV collagen (α3NC1), ADAMTS13, Desmoglein-1/3, or GPIb/IX, GPIIb/IIIa, GPIa/IIa, NMDA receptor, glutamic acid decarboxylase (GAD), amphiphysin and gangliosides GM1, GD3, GQ1B, MOG, SIRPα, CCR2, CSF-1R, LILRB1, LILRB2, VEGF-R, CXCR4, CCL2, CXCL12, CSF-1 or CD47.

In some embodiments, the bifunctional proteins binds to an endocytic carbohydrate-binding receptor, specifically binds to a mannose 3 receptor, a Cluster of Differentiation 206 (CD206) receptor, a DC-SIGN (Cluster of Differentiation 209 or CD209) receptor, a C-Type Lectin Domain Family 4 Member G (LSECTin) receptor, a macrophage inducible Ca²⁺-dependent lectin receptor (Mincle).

In some embodiments, the bifunctional protein binds to the endocytic carbohydrate-binding receptor via the N-glycan. In some embodiments, the N-glycan specifically binds to any endocytic carbohydrate-binding receptor recognizing Man3GlcNAc2 structure.

In some embodiments, the bifunctional binding protein is an antibody. In some embodiments, the antibody is a monoclonal or polyclonal antibody. In some embodiments, the antibody is recombinant. In some embodiments, the antibody comprises a heavy chain variable region or a light chain variable region. In some embodiments, the antibody comprises a Fab region. In some embodiments, the antibody comprises a Fc domain.

In some embodiments, the antibody comprises an N-glycosylation site in the Fc domain and wherein the N-glycan is linked to the N-glycosylation site in the Fc domain. In some embodiments, the antibody comprises an N-glycosylation site in the heavy chain variable regions and/or light chain variable regions and wherein the N-glycan is linked to the N-glycosylation site in the heavy chain variable regions and/or light chain variable regions.

In some embodiments, the antibody is glycosylated at a predetermined and specific residue.

In some embodiments, the antibody has a glycan to protein ratio of 2 to 1, 4 to 1, 6 to 1, 8 to 1, or 10 to 1.

In some embodiments, the bifunctional binding protein comprises an autoantigen and specifically binds to an autoantibody.

In some embodiments, the target protein is a cell surface molecule or a non-cell surface molecule. In some embodiments, the cell surface molecule is a receptor. In some embodiments, the non-cell surface molecule is an extracellular protein.

In some embodiments, the extracellular protein is an autoantibody, a hormone, a cytokine, a chemokine, a blood protein, or a central nervous system (CNS) protein.

In one aspect, provided herein is a method of delivering a target protein to liver macrophages: contacting the target protein with the bifunctional binding protein provided herein under conditions to mediate endocytosis of the target protein.

In one aspect, provided herein is a method of degrading a target protein comprising: contacting the target protein with the bifunctional binding protein provided herein under conditions to mediate lysosomal degradation of the target protein by a host cell.

In some embodiments, the target protein is upregulated in cancer or involved in cancer progression. In some embodiments, the target protein upregulated in cancer or involved in cancer progression comprises HER2, EGFR, HER3, VEGFR CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2, Frizzled receptors, Wnt, LRP5/6, CSF-1R, SIRPα, LILRB1, LILRB2, CD38, CD73, or TGF-β.

In some embodiments, the target protein is an autoantibody of an autoimmune disease. In some embodiments, the target protein is an autoantigen in an autoimmune disease. In some embodiments, the autoantibody in the autoimmune disease is an antibody binding to MOG, TSHRα, AChR-α1, noncollagen domain 1 of the α3 chain of type IV collagen (α3NC1), ADAMTS13, Desmoglein-1/3, GPIb/IX, GPIIb/IIIa, GPIa/Iia, NMDA receptor, glutamic acid decarboxylase (GAD), amphiphysin, or gangliosides GM1, GD3 or GQ1B.

In some embodiments, the target protein is upregulated or expressed in a neurodegenerative disease. In some embodiments, the target protein upregulated or expressed in a neurodegenerative disease is alpha-synuclein, amyloid beta or complement cascade component.

In some embodiments, the host cell is a myeloid cell, an immune cell, an endothelial cell, a parenchymal cell, or an epithelial cell. In some embodiments, the immune cell is a dendritic cell, a macrophage, a monocyte, a microglia cell, a granulocyte or a B lymphocyte. In some embodiments, the host cell is any cell.

In some embodiments, said bifunctional binding protein enhances degradation of the target protein relative to degradation of the target protein in the presence of a bifunctional binding protein without N-glycosylation or relative to degradation of the target protein in the presence of a bifunctional binding protein comprising an N-glycan different from a mannose 3 N-glycan structure. In some embodiments, said degradation is mediated by endocytosis or phagocytosis.

In one aspect, provided herein is a pharmaceutical composition comprising the bifunctional binding protein provided herein and a pharmaceutically acceptable carrier.

In one aspect, provided herein is a method of treating or preventing a disease in a patient comprising: administering to the patient the bifunctional binding protein provided herein, or the pharmaceutical composition provided herein.

In some embodiments, the disease is an autoimmune disease, a cancer or tumor, a liver disease, an inflammatory disorder, or a blood coagulation disorder. In some embodiments, the autoimmune disease is selected from MOGAD (Myelin oligodendrocyte glycoprotein antibody-associated disease), Graves' Disease, Myasthenia Gravis, Anti-GBM Disease, Immune Thrombotic Thrombocytopenic Purpura, Acquired Pemphigus Vulgaris, Immune Thrombocytopenia, autoimmune encephalitis, and Guillain-Barre Syndrome.

In some embodiments, the cancer is selected from acute lymphoblastic leukemia; acute lymphoblastic lymphoma; acute lymphocytic leukaemia; acute myelogenous leukemia; acute myeloid leukemia (adult/childhood); adrenocortical carcinoma; AIDS-related cancers; AIDS-related lymphoma; anal cancer; appendix cancer; astrocytomas; atypical teratoid/rhabdoid tumor; basal-cell carcinoma; bile duct cancer, extrahepatic (cholangiocarcinoma); bladder cancer; bone osteosarcoma/malignant fibrous histiocytoma; brain cancer (adult/childhood); brain tumor, cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; ewing's sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver) cancer; hodgkin lymphoma; hypopharyngeal cancer; hypothalamic and visual pathway glioma; intraocular melanoma; islet cell carcinoma (endocrine pancreas); Kaposi Sarcoma; kidney cancer (renal cell cancer); langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); merkel cell cancer; merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, nonsmall cell lung cancer; non-hodgkin lymophoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; respiratory tract carcinoma involving the NUT gene on chromosome 15; retinoblastoma; rhabdomyosarcoma, childhood; salivary gland cancer; sarcoma, Ewing family of tumors; Sezary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sezary syndrome); testicular cancer; throat cancer; thymoma; thymoma and thymic carcinoma; thyroid cancer; childhood thyroid cancer; transitional cell cancer of the renal pelvis and ureter; urethral cancer; uterine cancer, endometrial; uterine sarcoma; vaginal cancer; vulvar cancer; or Wilms tumor.

In some embodiments, the treatment comprises reprogramming tumor associated macrophages (TAMs) by administering the bifunctional binding protein under conditions to mediate endocytosis of a target protein.

In some embodiments, the target protein is upregulated or expressed in TAMs. In some embodiments, the target protein upregulated or expressed in TAMs comprises SIRPα, CCR2, CSF-1R, LILRB1, LILRB2, VEGF-R, CXCR4, CCL2, CXCL12, CSF-1 or CD47.

In some embodiments, the administration step comprises intravenous injection, intraperitoneal injection, subcutaneous injection, transdermal injection, or intramuscular injection.

In one aspect, provided herein is a kit comprising the bifunctional binding protein provided herein, or the pharmaceutical composition provided herein and instructions for administering the bifunctional molecule or pharmaceutical composition to an individual in need thereof.

In some embodiments, the bifunctional binding protein or pharmaceutical composition is present in one or more unit dosages.

In one aspect, provided herein is a method of treating an acute condition associated with increased levels of a target protein, wherein the method comprises administering to a patient in need thereof a bifunctional binding protein of any one of the preceding claims, wherein the bifunctional binding protein (i) specifically binds to the target protein and (ii) comprises an N-glycan of the structure

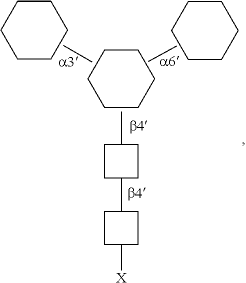

wherein the square represents an N-acetylglucosamine residue and the hexagon represents a mannose residue, and wherein X represents an amino acid residue of the bifunctional binding protein, wherein the N-glycan is linked to the bifunctional binding protein at a number of N-glycosylation sites that results in a half-life of the target protein of at most 5 minutes, 15 minutes, 30 minutes, 1 hour, 2 hours, 3 hours, 4 hours, 5 hours, or 6 hours, or that results in a half-life of the target protein of at most 1%, 5%, 10%, 15%, 20%, 25%, 30%, 35%, 40%, 45%, 50%, 60%, 70%, 75%, 80%, or at most 90% of the half-life of the target protein in the patient in the absence of any treatment.

In some embodiments, the glycan further comprises a fucose residue at the N-acetylglusoamine that is directly attached to X. In some embodiments, X is an asparagine residue in the bifunctional binding protein.

In some embodiments, the bifunctional protein carries the N-glycan at three or more N-glycosylation sites.

In one aspect, provided herein is a method of treating a chronic condition associated with increased levels of a target protein, wherein the method comprises administering to a patient in need thereof a bifunctional binding protein of any one of the preceding claims, wherein the bifunctional binding protein (i) specifically binds to the target protein and (ii) comprises an N-glycan of the structure

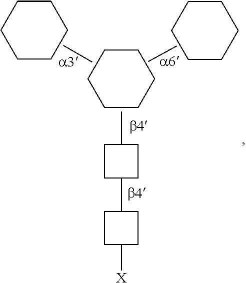

wherein the square represents an N-acetylglucosamine residue and the hexagon represents a mannose residue, and wherein X represents an amino acid residue of the bifunctional binding protein, wherein the N-glycan is linked to the bifunctional binding protein at a number of N-glycosylation sites that results in a half-life of the target protein of at least 1 day, 2 days, 3 days, or 4 days, or in a half-life of the target protein of at least 50%, 55%, 60%, 65%, 70%, 75%, 80%, 85%, 90%, or at least 95% of the half-life of the bifunctional binding protein without glycosylation in the patient.

In some embodiments, the glycan further comprises a fucose residue at the N-acetylglusoamine that is directly attached to X. In some embodiments, X is an asparagine residue in the bifunctional binding protein.

In some embodiments, the bifunctional protein carries the N-glycan at two or less N-glycosylation sites.

### 5. DESCRIPTION OF THE DRAWINGS

FIG. 1 shows that Man3 glycan displayed on Fab are highly internalized by macrophages. Macrophages were obtained and incubated for 3 hours with pHrodo-labeled antibodies. The graph shows the average mean fluorescence intensity (MFI) of pHrodo normalized to Humira (H-A2F)±standard error of the mean (SEM). Each point shows the value for an individual donor. At least 3 donors for each condition except for H-PNGase (N=2).

FIG. 2 Man3 Glycan on an antibody leads to high internalization and lysosomal compartment targeting in human dendritic cells. Internalization is independent from Fcγ receptor. Human monocyte-derived dendritic cells were obtained from 5 different PBMC donors. The graph shows the average pHrodo MFI±standard deviation (SD) from N=5 individual donors. No Ab indicates that no pHrodo-labeled antibody was added whereas all other conditions indicate pHrodo-labeled antibody added at 10 g/ml. MbT: Mabthera used as control antibody. A-M: Man3-adalimumab. A-S: Sialylated adalimumab. H-M: HUMIRA® (adalimumab). H-S: sialylated HUMIRA® (adalimumab). +FcR blocking indicate that the Fcγ receptor blocking agent Human TruStain FcX was added before pHrodo-labeled antibodies were added.

FIGS. 3A and 3B show that Man3 glycan displayed on Fab are highly internalized by dendritic cells. Dendritic cells were obtained from 2 PBMC donors. Dendritic cells were incubated for 6 hours with pHrodo-labeled antibodies. FIG. 3A: Example of gating the pHrodo High population for the condition H-A2F and A8486-M3. The pHrodo High gated cells are in black. FIG. 3B: the graph shows the percentage of pHrodo High cells (gated as indicated in FIG. 3A) for each donor.

FIG. 4 Schematic representation that mannose 3 is recognized by mannose binding receptors and lectins such as CD206.

FIG. 5 shows the phenotype of the macrophages after differentiation. Macrophages were CD14+CD163+CD1a−CD206+. The flow cytometry plot shows the marker expression from one representative culture. The graph on the right shows that the percentage of CD206+ macrophages is consistent between donors and experiments. Each point represents the data from one peripheral blood mononuclear cell (PBMC) donor.

FIGS. 6A and 6B show that antibodies displaying M3 structure on Fab are specifically internalized by CD206 expressing macrophages. Macrophages were incubated for 3 hours (A) or 24 hours (B) with pHrodo-labeled antibodies. The graphs show the pHrodo MFI adjusted to degree of labeling (DOL). The histograms show the average±SD from all donors and each dot show data from 1 donor. Statistical analysis Two-tailed paired t-test. *p<0.05; **p<0.01; ***p<0.001; ns: not significant (p>0.05).

FIG. 7 shows that antibodies displaying Man3 glycan structure leads to potent and rapid elimination of a target antigen from blood circulation in rat. Rats were injected intravenously (i.v.) with HCA202 (0.5 mg/kg) and with antibodies i.v. 10 mg/kg. Graph shows average±SD of HCA202 serum concentration in ng/ml of 3 or 4 animal/group. Black circles show H-A2F (adalimumab, Humira®) treated group. Open squares show A-84-M3 treated group. Black triangles show A-8486-M3 treated group. Black square show A-8486-A2G2S2 treated group. Open circles show PBS treated group (HCA202 only). Open diamonds and dotted line show A-M3 treated group. For graphical representation, when HCA202 levels were below assay Lower Limit of Quantification (LLOQ) at the minimal required 10 fold dilution (MRD10) (dotted LLOQ/MRD10 line=20 ng/ml), they were set at 19 ng/ml.

FIG. 8 shows that an antibody displaying Man3 glycan structure is distributed partially to the liver area with a fast kinetic as compared to control antibodies. Mice were injected i.v. with CF750-labeled antibodies at 5 mg/kg and imaged using fluorescence tomography. The graph shows the average fluorescence in pmol±SD of 3 animals/timepoint in the gated liver region of interest. Open Squares and dotted line show A-M3 treated group. Black Circles show H-A2F treated group. Black triangles show A-8486-M3 treated group.

### 6. DETAILED DESCRIPTION OF THE INVENTION

Described herein is bifunctional binding protein (e.g., a mannose 3 glycosylated bifunctional binding protein) having improved functionalities as compared to a control antibody. As exemplified herein, the bifunctional binding protein is engineered by introduction of mannose 3 on glycosylation sites on the bifunctional binding protein, resulting in an engineered glycosylation profile that mediates endocytic receptor degradation of the bifunctional binding protein and the target to which it binds. By customizing the mannose 3 glycan site, the engineered bifunctional binding proteins described herein: 1) have homogeneous glycosylation; 2) can degrade large targets such as immune complexes; 3) have a defined ligand-to-antibody ratio; 4) have defined glycosylation sites; 6) can activate more diverse and powerful degradation receptors; and/or 5) can engage in protein degradation in a highly optimized manner.

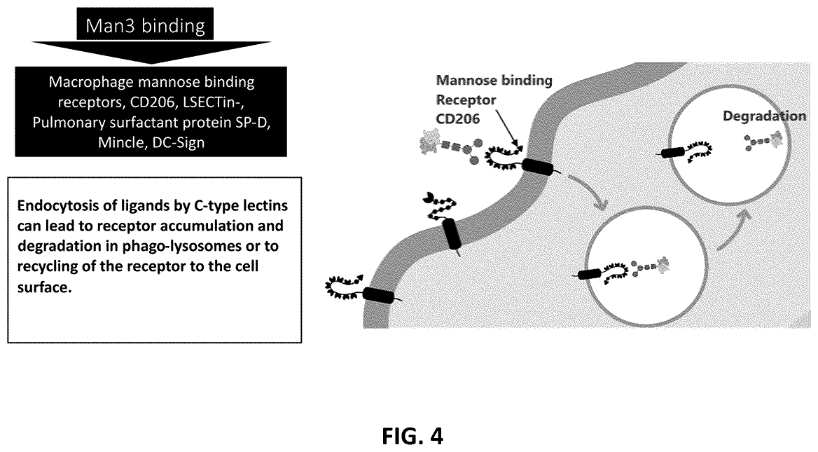

As shown in FIG. 4, without being bound by theory, the bifunctional binding protein, as described herein, can bind to macrophage mannose binding receptors, CD206, LSECTin-, Pulmonary surfactant protein SP-D, Mincle, DC-Sign, and any other lectin recognizing mannose 3. Endocytosis of ligands by C-type lectins can lead to receptor accumulation and degradation in phago-lysosomes or to recycling of the receptor to the cell surface.

Without being bound by theory, the mannose 3 glycosylated antibody, as described herein, is expected to reduce target proteins associated with human disease, via natural degradation pathways.

The term “about,” when used in conjunction with a number, refers to any number within ±1, ±5 or +10% of the referenced number.

As used herein, the term “patient” refers to an animal (e.g., birds, reptiles, and mammals). In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, a subject is a non-human animal. In some embodiments, a subject is a farm animal or pet (e.g., a dog, cat, horse, goat, sheep, pig, donkey, or chicken). In a specific embodiment, a subject is a human. The terms “subject” and “patient” can be used herein interchangeably.

The abbreviations “α[number]”, “α[number], [number]”, “β[number]”, or “β[number], [number]” refer to glycosidic bonds or glycosidic linkages which are covalent bonds that join a carbohydrate residue to another group. An α-glycosidic bond is formed when both carbons have the same stereochemistry, whereas a β-glycosidic bond occurs when the two carbons have different stereochemistry.

As used herein, the term “bifunctional binding protein” means a protein capable of binding two different ligands via a first moiety and a second moiety, wherein the first moiety is different from the second moiety. As used herein the term “bifunctional binding protein” does refer to proteins with more than two binding specificities or functions. An example of a bifunctional binding protein of the present disclosure can include a mannose 3 glycosylated anti-TSH receptor antibody, or a mannose 3 glycosylated soluble TSH receptor ectodomain binding to autoantibodies.

As used herein, the term “inflammatory disorder” includes disorders, diseases or conditions characterized by inflammation. Examples of inflammatory disorders include allergy, asthma, autoimmune diseases, coeliac disease, glomerulonephritis, hepatitis, inflammatory bowel disease, reperfusion injury and transplant rejection, among others.

As used herein, the term “blood disorder” includes a disorders, diseases or conditions that affect blood. Examples of blood disorders include anemia, bleeding disorders such as hemophilia, blood clots, and blood cancers such as leukemia, lymphoma, and myeloma, among others.

As used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier,” as used herein in the context of a pharmaceutically acceptable carrier, refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin.

In some embodiments, a bifunctional binding protein provided herein can comprise (i) a binding specificity to one or more target protein(s) and (ii) one or more N-glycan(s) with binding specificities to one or more endocytic carbohydrate-binding protein or receptor.

In some embodiments, the glycoengineered bifunctional binding protein has one binding specificity to one target protein. In more specific embodiments, the glycoengineered bifunctional binding protein has one binding specificity to one target protein and has 1, 2, 3, 4, 5, 6, 7, 8, 9, 10 or more valences so that a single bifunctional binding protein can bind to 1, 2, 3, 4, 5, 6, 7, 8, 9, 10 or more target protein molecules.

In some embodiments, the bifunctional binding protein has one or more N-glycan(s) capable of engaging an endocytic receptor. In some embodiments, the bifunctional binding protein has two, three, four, five, six, seven, eight, nine, ten or more N-glycans capable of engaging an endocytic receptor. The N-glycans can be linked to the protein at one, two, three, four, five, six, seven, eight, nine, ten or more N-glycosylation consensus sequences (or glycosites). These glycosites can be naturally occurring in the protein or recombinantly introduced via amino acid additions, substitutions, or deletions. In a specific embodiment, a glycotag is fused to a protein to create a bispecific binding protein. As used herein a glycotag refers to a peptide containing consensus N-glycosylation site sequence fused to N- or a C-terminal or both termini of a protein or polypeptide. In some embodiments, the two or more, three or more, four or more, five or more, six or more, seven or more, eight or more, nine or more or ten or more glycans are the same and engage the same endocytic receptor(s). In some embodiments, the two or more, three or more, four or more, five or more, six or more, seven or more, eight or more, nine or more or ten or more glycans are different and engage different endocytic receptors.

In some embodiments, provided herein is a bifunctional binding protein that specifically binds to a target protein, comprising a first moiety and a second moiety.

In some embodiments, the first moiety comprises a heavy chain variable region and a light chain variable region, or an antigenic fragment thereof. In some embodiments, the first moiety comprises a Fab region of a monoclonal antibody. In some embodiments, the first moiety specifically binds to any of the target proteins disclosed herein.

In some embodiments, the bifunctional binding protein is a TNFα monoclonal antibody. In other embodiments, the bifunctional binding protein is not a TNFα monoclonal antibody.

In some embodiments, the second moiety comprises a glycan structure having a mannose 3 glycan structure as disclosed herein. In some embodiments, the glycan structure is any of the glycans disclosed herein.

In some embodiments, any receptor that binds a mannose 3 glycan is included in the description of the compositions and methods disclosed herein. In other embodiments, the second moiety binds to any endocytic carbohydrate-binding receptor recognizing Man3GlcNAc2 structure.

In some embodiments, the second moiety specifically binds to a mannose 3 receptor. In some embodiments, the second moiety specifically binds to the Cluster of Differentiation 206 (CD206) receptor. In some embodiments, the second moiety specifically binds to the DC-SIGN (Cluster of Differentiation 209 or CD209) receptor. In some embodiments, the second moiety specifically binds to the C-Type Lectin Domain Family 4 Member G (LSECTin) receptor. In some embodiments, the second moiety specifically binds to the macrophage inducible Ca²⁺-dependent lectin receptor (Mincle). In some embodiments, the second moiety specifically binds to a receptor selected from the group consisting of langerin, a macrophage mannose 2 receptor, dectin-1 dectin-2, BDCA-2, DCIR, MBL, MDL, MICL, CLEC2, DNGR1, CLEC12B, DEC-205, asialoglycoprotein receptor (ASPGR), and mannose 6 phosphate receptor (M6PR).

In some embodiments, the bifunctional binding protein is an antibody. In some embodiments, the antibody is a monoclonal antibody, a polyclonal antibody, or an antigenic fragment thereof. In some embodiments, the antibody is a recombinant antibody. In some embodiments, the antibody is isolated from a human subject. In some embodiments, the antibody is humanized, chimeric or fully human. In other embodiments, the bifunctional binding protein is an autoantigen. In other embodiments, the bifunctional binding protein is an autoantibody.

In some embodiments, the antibody has a glycan to antibody ratio of 2 to 1, 4 to 1, 6 to 1, 8 to 1 or 10 to 1. In some embodiments, the antibody is glycosylated at a predetermined and specific residue. In other embodiments, the antibody is glycosylated at a random residue.

In some embodiments, provided herein is a bifunctional binding protein comprising a second moiety with the following structure:

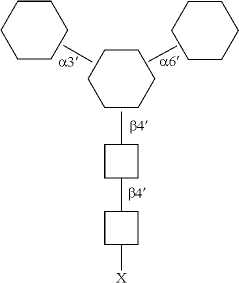

wherein the square represents an N-acetylglucosamine residue and the hexagon represents a mannose residue, and wherein X represents an amino acid residue of the bifunctional binding protein. In some embodiments, the X amino acid residue of the bifunctional binding protein as disclosed herein is an Asn of an N-linked glycosylation consensus sequence that is engineered into the bifunctional binding protein. Such an N-linked glycosylation consensus sequence can be, in some embodiments, in a variable domain of the bifunctional binding protein.

In some embodiments, the bifunctional binding protein comprises at least 20%, 25%, 30%, 35%, 40%, 45%, 50%, 55%, 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of the glycans having the structure of

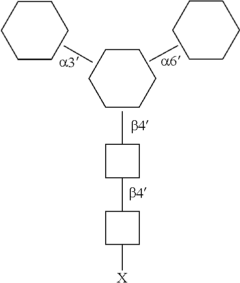

wherein X represents an amino acid residue of the bifunctional binding protein. In some embodiments, the X amino acid residue of the bifunctional binding protein disclosed herein is an Asn of an N-linked glycosylation consensus sequence that is engineered into the bifunctional binding protein.

In some embodiments, the bifunctional binding protein is an anti-TGF-β monoclonal antibody or an anti-Notch monoclonal antibody glycoengineered with Mannotriose-di-(N-acetyl-D-glucosamine) (Man3GlcNAc2).

In some embodiments, the target protein is a cell surface molecule or a non-cell surface molecule. In some embodiments, the cell surface molecule is a receptor. In some embodiments, the non-cell surface receptor is an extracellular protein. In some embodiments, the extracellular protein is an autoantibody, a hormone, a cytokine, a chemokine, a blood protein, or a protein expressed in the central nervous system (CNS).

In some embodiments, the target protein associated with a disease is upregulated in the disease compared to a non-disease state. In some embodiments, the target protein associated with a disease is expressed in the disease compared to a non-disease state. In some embodiments, the target protein associated with a disease is involved in the progression of the disease. In some embodiments, the disease is a cancer or tumor. In some embodiments the disease is an autoimmune disease. In some embodiments, the disease is neurodegenerative disease.

In some embodiments, the disease is Graves' disease. Graves' disease is the most common cause of hyperthyroidism. Prevalence in the US is 1.2% (1), with lifetime risk in women as high as 3%. Production of agonistic anti-TSH Receptor (TSHR) antibodies (TRAb) leading to over production of thyroxine hormone (>90% of patients are TRAb+) (2). Current treatments have not advanced in the 50 years and are limited by high risk of recurrence or severe side effects such as hypothyroidism. In some embodiments, the target protein associated with Graves' disease is an autoantibody binding TSHRα. In other embodiments, the target protein associated with Graves' disease is TSHRα.

In some embodiments, the target protein comprises a protein selected from the group consisting of HER2, EGFR, HER3, VEGFR, CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2 Frizzled receptors, Wnt, LRP5/6, CSF-1R, SIRPα, LILRB1, LILRB2, LILRB3, LILRB4, CD38, CD73, TGF-β. In other embodiments, the target protein comprises an antibody that binds to TSHRα, MOG, AChR-α1, noncollagen domain 1 of the α3 chain of type IV collagen (α3NC1), ADAMTS13, Desmoglein-1/3, GPIb/IX, GPIIb/IIIa, GPIa/IIa, NMDA receptor, glutamic acid decarboxylase (GAD), amphiphysin and gangliosides GM1, GD3, and GQIB.

In some embodiments, a method of delivering a target protein to a hepatocyte endosome is provided herein. In some embodiments, the method of delivering a target protein to a hepatocyte comprises contacting the target protein with any of the bifunctional binding proteins disclosed herein under conditions to mediate endocytosis of any of the target proteins disclosed herein. In some embodiments, the method of delivering the target protein to an endosome of a hepatic macrophage, such as liver resident Kupffer cells (KCs) and/or liver sinusoidal endothelial cells (LSEC) and/or monocyte-derived macrophages (MoMϕs), occurs in vivo. In some embodiments, the mode of delivering a target protein to a hepatocyte endosome in vivo comprises intravenous injection, intraperitoneal injection, subcutaneous injection, transdermal injection or intramuscular injection. In some embodiments, the method of delivering the target protein to a hepatocyte endosome occurs ex vivo.

In some embodiments, a method of degrading a target protein is provided herein. In some embodiments, the method of degrading a target protein comprises contacting the target protein with any of the bifunctional binding proteins disclosed herein under conditions to mediate degradation of any of the target proteins disclosed herein by a host cell. In some embodiments, degradation is lysosomal degradation. In some embodiments, degradation is mediated by endocytosis or phagocytosis. In some embodiments, degradation is at least 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than degradation mediated by a bifunctional binding protein comprising a glycan other than any of the second moieties disclosed herein. In other embodiments, the bifunctional binding protein enhances degradation of any of the disclosed target proteins relative to degradation of the target protein in the presence of a bifunctional binding protein comprising a glycan other than any of the second moieties disclosed herein. In some embodiments, the host cell is any host cell, including, but not limited to, a myeloid cell, an immune cell, an endothelial cell, a parenchymal cell or an epithelial cell. In some embodiments, the immune cell can be a dendritic cell, a macrophage, a monocyte, a microglia cell, a granulocyte or a B lymphocyte.

In some embodiments, mannose 3 degradation is optimal due to engagement of endocytic receptors. In some embodiments, the method of degrading a target protein via mannose 3 mediated degradation is selective. In some embodiments, mannose 3 degradation removes inflammatory cytokines from circulation, removes unwanted blood factors, removes autoantibodies and removes cell surface receptors.

In some embodiments, the bifunctional binding protein that mediates degradation of a target protein is an anti-TGF-βmonoclonal antibody comprising a Man3GlcNAc2 that captures TGF-β and degrades TGF-β in the lysosome via recognition of the Man3GlcNAc2 by any of the endocytic receptors disclosed herein. This approach can be applied to deplete a cell surface a receptor such Notch for cancer treatment.

In some embodiments, the target protein is a protein that is upregulated in cancer. In some embodiments, the target protein is a protein that is involved in cancer progression. Examples of target proteins that are upregulated in cancer or involved in cancer progression that can be bound by a bifunctional binding protein provided herein include, but are not limited to, HER2, EGFR, HER3, VEGFR, CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2, Frizzled receptors, Wnt, LRP5/6, CSF-1R, SIRPα, LILRB1, LILRB2, LILRB3, LILRB4, CD38, CD73, TGF-β, Bombesin R, CAIX, CD13, CD44v6, CXCR4, ErbB-2, Her2, Emmprin, Endoglin, EpCAM, EphA2, FAP-α, Folate R, GRP78, IGF-1R, Matriptase, Mesothelin, sMET/HGFR, MT1-MMP, MT6-MMP, Muc-1, PSCA, PSMA, Tn antigen, and uPAR.

In some embodiments, the target protein is an autoantibody, such as those associated with an autoimmune disease. Examples of an autoantibody that can be bound by a bifunctional binding protein provided herein include, but are not limited to, autoantibodies directed against MOG, TSHRα, AChR-α1, noncollagen domain 1 of the α3 chain of type IV collagen (α3NC1), ADAMTS13, Desmoglein-1/3, or GPIb/IX, GPIIb/IIIa, GPIa/IIa, NMDA receptor, glutamic acid decarboxylase (GAD), amphiphysin and gangliosides GM1, GD3, GQ1B.

In some embodiments, the target protein comprises a protein that is upregulated or expressed in tumor associated macrophages (TAMs). In some embodiments, the target protein is upregulated or expressed in pro-tumorigenic TAMs. Examples of target proteins upregulated or expressed in TAMs comprise SIRPα, CCR2, CSF-1R, LILRB1, LILRB2, VEGF-R, or CXCR4 (7). In other embodiments, the target proteins comprise CCL2, CXCL12, CSF-1 or CD47 (7). These targets, as described in reference (7), play a role in promoting pro-tumor TAMs particularly by promoting TAM recruitment and programming.

In some embodiments, provided herein is a method of reprogramming TAMs using any of the bifunctional binding proteins disclosed herein. As described in reference (7), reprogramming TAMs comprises targeting and inhibiting macrophage receptors to reprogram pro-tumorigenic TAMs into anti-tumorigenic TAMs. In other embodiments, provided herein is a method of depleting TAMs using any of the bifunctional binding proteins disclosed herein. Depletion of TAMs can occur by targeting receptors important for proliferation. Targeting of these receptors can promote apoptosis of pro-tumorigenic TAMs. In further embodiments, provided herein is a method of inhibiting recruitment of TAMs to the tumor microenvironment, using any of the bifunctional binding proteins disclosed herein. In some embodiments, the method of reprogramming, depleting or inhibiting recruitment of TAMs comprises using any of the disclosed bifunctional binding proteins to degrade a target upregulated or expressed by TAMs. In some embodiments, the target protein upregulated or expressed in TAMs comprises SIRPα, CCR2, CSF-1R, LILRB1, LILRB2, VEGF-R, or CXCR4. In other embodiments, the target protein associated with TAMs comprises CCL2, CXCL12, CSF-1 or CD47.

In some embodiments, provided herein is a pharmaceutical composition comprising the bifunctional binding protein described herein and a pharmaceutically acceptable carrier.

In some embodiments, provided herein is a method of treating or preventing a disease in a patient comprising administering to the patient a bifunctional binding protein described herein or a pharmaceutical composition described herein. In some embodiments, the disease is an autoimmune disease, a cancer or tumor, a liver disease, an inflammatory disorder, or a blood disorder. In some embodiments, the autoimmune disease is selected from Graves' Disease, Myelin oligodendrocyte glycoprotein antibody-associated disease (MOGAD), membranous nephropathy, Myasthenia Gravis, Anti-GBM Disease, Immune Thrombotic Thrombocytopenic Purpura, Acquired Pemphigus Vulgaris, Immune Thrombocytopenia, and Guillain-Barre Syndrome. In some embodiments, the cancer or tumor is selected from breast cancer, colorectal cancer, pancreatic cancer, non-small cell lung cancer, hepatocellular carcinoma, and hematological T cell and B cell malignancies.

In some embodiments, a method of treating or preventing a disease provided herein includes an administration step that comprises intravenous injection, intraperitoneal injection, subcutaneous injection, transdermal injection, or intramuscular injection of a bifunctional binding protein described herein or a pharmaceutical composition described herein.

In some embodiments, a method of treating or preventing a disease provided herein requires a lower dose and/or lower administration frequency to achieve the same effect as compared to the same antibody having a different glycosylation profile; and/or can be administered for an extended period of time (at least 1, 2, 3, 4, 5, 6, 7, 8, 9, 10, 11 or at least 12 months, at least 2, 3, 4, 5, 6, 7, 8, 9, or at least 10 years); and/or does not trigger an immune response against the bifunctional binding protein in the patient.

In some embodiments, provided herein is a kit comprising the bifunctional binding protein of the present disclosure is provided herein. In some embodiments, the kit further provides instructions for administering the bifunctional molecule or pharmaceutical composition to an individual in need thereof.

In some embodiments, the pharmaceutical compositions described herein can be administered in a single dosage form, for example a single dosage form of a bifunctional binding protein described here.

In some embodiments, a suitable dose of a bifunctional binding protein described herein is the amount corresponding to the lowest dose effective to produce a therapeutic effect. For example, an effective amount of an anti-TSH receptor antibody can be an amount that inhibits TSH activity in a subject suffering from a Graves' disease.

In some embodiments, the amount of bifunctional binding protein described herein administered to a patient is less than the amount listed in the label of a drug product of the same bifunctional binding protein having no glycosylation profile or a different glycosylation profile from that of the bifunctional binding protein described herein.

In some embodiments, the accumulated amount of a bifunctional binding protein described herein administered to a patient over a period of time is less than the accumulated amount indicated in the label of a drug product of the same bifunctional binding protein having no glycosylation profile or a different glycosylation profile from that of the bifunctional binding protein described herein. In some embodiments, the reduced accumulated amount could be administered in reduced doses on a reduced frequency. In some embodiments, the reduced accumulated amount could be administered in one or more doses that are the same or higher than the dose in the label on a reduced frequency. In some embodiments, the reduced accumulated amount could be administered in one or more reduced doses on a frequency that is the same or higher than the frequency in the label. In some embodiments, the reduced accumulated amount could be administered over a shorter period of time than the period of time for the drug product to achieve the same level of effect in treatment or prevention.

In some embodiments, the amount of the bifunctional binding protein described herein in a single dose administered to a patient can be from about 1 to 150 mg, about 5 to 145 mg, about 10 to 140 mg, about 15 to 135 mg, about 20 to 130 mg, about 25 to 125 mg, about 30 to 120 mg, about 35 to 115 mg, about 40 to 110 mg, about 45 to 105 mg, about 50 to 100 mg, about 55 to 95 mg, about 60 to 90 mg, about 65 to 5 mg, about 70 to 80 mg, or about 75 mg. In some embodiments, the amount of bifunctional binding protein described herein in a single dose administered to a patient can be from about 5 to about 80 mg. In some embodiments, the amount of bifunctional binding protein described herein in a single dose administered to a patient can be from about 25 to about 50 mg. In some embodiments, the amount of a bifunctional binding protein described herein in a single dose administered to a patient can from about 15 mg to about 35 mg.

In some embodiments, the amount of a bifunctional binding protein described herein in a single dose administered to a patient can be no more than 40 mg, for example 40 mg, 35 mg, 30 mg, 25 mg, 20 mg, 18 mg, 15 mg, 12 mg, 10 mg, 7 mg, 5 mg, and 2 mg. In some embodiments, the amount of a bifunctional binding protein described herein in a single dose administered to a patient can be no more than 80 mg, for example 80 mg, 75 mg, 70 mg, 65 mg, 60 mg, 55 mg, 50 mg, 45 mg, 40 mg, 35 mg, 30 mg, 20 mg, 15 mg, 10 mg, 5 mg and 2 mg. In some embodiments, the amount of a bifunctional binding protein described herein in a single dose administered to a patient can be no more than 160 mg, for example 150 mg, 140 mg, 130 mg, 120 mg, 110 mg, 100 mg, 90 mg, 80 mg, 75 mg, 70 mg, 65 mg, 60 mg, 55 mg, 50 mg, 45 mg, 40 mg, 35 mg, 30 mg, 20 mg, 15 mg, 10 mg, 5 mg and 2 mg. In some embodiments, the amount of a bifunctional binding protein described herein in a single dose administered to a patient can be equal to or more than 160 mg, for example 170 mg, 180 mg, 200 mg, 250 mg, and 300 mg.

In some embodiments, a bifunctional binding protein of the disclosure can be administered on a frequency that is every other week, namely every 14 days. In some embodiments, a bifunctional binding protein of the disclosure can be administered on a frequency that is lower than every 14 days, for example, every half a month, every 21 days, monthly, every 8 weeks, bimonthly, every 12 weeks, every 3 months, every 4 months, every 5 months, or every 6 months. In some embodiments, a bifunctional binding protein of the disclosure can be administered on a frequency that is the same or higher than every 14 days, for example, every 14 days, every 10 days, every 7 days, every 5 days, every other day, or daily.

In some embodiments, the administration of a bifunctional binding protein of the disclosure can comprise an induction dose that is higher than the following doses, for example the following maintenance doses. In some embodiments, the administration of a bifunctional binding protein of the disclosure can comprise a second dose that is lower than the induction dose and higher than the following maintenance doses. In some embodiments, the administration of a bifunctional binding protein of the disclosure can comprise the same amount of the bifunctional binding protein in all the doses throughout the treatment period.

Methods of generating a bifunctional binding protein provided herein are well known in the art. Exemplary methods of generating a bifunctional binding protein provided herein are described in International Patent Application Publications WO2019/002512, WO2021/140143, WO2021/140144, and WO2022/053673, which are incorporated herein by reference in their entirety, and are exemplified herein, any one of which can be used to generate a bifunctional binding protein provided herein. For example, one of skill in the art will readily appreciate that the nucleic acid sequence of a known protein (e.g., a monoclonal antibody), as well as a newly identified protein (e.g., a monoclonal antibody), can easily be deduced using methods known in the art, and thus it would be well within the capacity of one of skill in the art to introduce a nucleic acid that encodes any bifunctional binding protein into a host cell provided herein (e.g., via an expression vector, e.g., a plasmid, e.g., a site specific integration by homologous recombination).

In some embodiments, provided herein is a Leishmania host cell comprising the bifunctional binding protein described herein. Such a host cell, in some embodiments, is Leishmania tarentolae. In some embodiments, the host cell is a Leishmania aethiopica cell. In some embodiments, the host cell is part of the Leishmania aethiopica species complex. In some embodiments, the host cell is a Leishmania aristidesi cell. In some embodiments, the host cell is a Leishmania deanei cell. In some embodiments, the host cell is part of the Leishmania donovani species complex. In some embodiments, the host cell is a Leishmania donovani cell. In some embodiments, the host cell is a Leishmania chagasi cell. In some embodiments, the host cell is a Leishmania infantum cell. In some embodiments, the host cell is a Leishmania hertigi cell. In some embodiments, the host cell is part of the Leishmania major species complex. In some embodiments, the host cell is a Leishmania major cell. In some embodiments, the host cell is a Leishmania martiniquensis cell. In some embodiments, the host cell is part of the Leishmania mexicana species complex. In some embodiments, the host cell is a Leishmania mexicana cell. In some embodiments, the host cell is a Leishmania pifanoi cell. In some embodiments, the host cell is part of the Leishmania tropica species complex. In some embodiments, the host cell is a Leishmania tropica cell.

In some embodiments, provided herein is a method for making a bifunctional binding protein comprising culturing a Leishmania host cell described herein and isolating the bifunctional binding protein.

In some embodiments, provided herein is a bifunctional binding protein produced by the method described herein. Methods of producing a Leishmania host cell and using such host cells to produce a bifunctional binding protein are well known in the art. Exemplary methods are described in International Patent Application Publications WO 2019/002512, WO2021/140143, WO2021/140144, and WO2022/053673, which are incorporated herein by reference in their entirety, and are exemplified herein, any one of which can be used to generate a Leishmania host cell and produce a bifunctional binding protein provided here. For example, in some embodiments, host cells described herein are cultured using any of the standard culturing techniques known in the art, including, but not limited to, growth in rich media like Brain Heart Infusion, Trypticase Soy Broth or Yeast Extract, all containing 5 μg/ml Hemin. Additionally, incubation can be done at 26° C. in the dark as static or shaking cultures for 2-3 days. In some embodiments, cultures of host cell contain the appropriate selective agents. For example, in some embodiments, monoclonal antibody described herein is purified from host cell culture supernatants using any of the standard purification techniques known in the art, including, but not limited to, Protein A Affinity Chromatography, Ion Exchange Chromatography and Mixed Mode Chromatography.

In some embodiment, an N-glycosylation site is an N-linked glycosylation consensus sequence. In certain embodiment, the N-linked glycosylation consensus sequence can be one or more asparagine residues. In further embodiment, the N-linked glycosylation consensus sequence can be one or more asparagine residues that fall within a canonical consensus sequence N-X-S/T, N-X-C motifs, and non-canonical consensus motifs.

In some embodiment, the N-glycosylation site is engineered into the bifunctional protein. In some embodiment, the N-glycosylation site can be introduced by inserting the N-linked glycosylation consensus sequence into a sequence of the bifunctional protein. In some embodiment, one or more sequences encoding N-linked glycosylation consensus sequence can inserted into the bifunctional protein sequence at one or more sites. In some embodiment, the insertion of the N-linked glycosylation consensus sequence can be performed by molecular biology techniques, for example, DNA cloning, extraction of DNA, bacterial transformation, transfection, chromosome integration, cellular screening, cellular culture, DNA sequencing, DNA synthesis, and molecular hybridization. In some embodiment, the N-glycosylation site can be introduced by nuclease-based precise gene editing tools, for example, zinc-finger nucleases, transcription activator-like effector nucleases (TALENs), and clustered regularly interspaced short palindromic repeat/targeted Cas9 endonuclease (CRISPR/Cas9).

In some embodiment, the bifunctional protein is an antibody. In some embodiment, the N-linked glycosylation consensus sequence can be introduced into one or more sites on Fab of the antibody. In some embodiment, the N-linked glycosylation consensus sequence can be introduced into one or more sites on Fc of the antibody.

In some embodiment, the bifunctional protein is adjusted to a condition by changing the number of glycosites, thus optimizing the potency and kinetics for the respective condition. In certain embodiment, 1, 2, 3, 4, 5, 6, 7, 8, 9, or 10 glycosites are engineered into Fab or Fc of the antibody. In certain embodiment, one engineered glycosite displaying Man3 glycan on one Fab shows low potency and kinetics of macrophage internalization, and two, three, four, or five engineered glycosites displaying Man3 glycans on one Fab show fast potency or kinetics of macrophage internalization. In certain embodiment, one engineered glycosite displaying M3 glycan on one Fab shows low potency or kinetics of target protein degradation, and two, three, four, or five engineered glycosites displaying M3 glycans on one Fab show fast potency or kinetics of target protein degradation. In some embodiment, the bifunctional protein comprises glycosites that are different from Man3 glycan.

### Method for Making Man3 Carrying Proteins

Methods of producing recombinant proteins are known in the art. Methods of bioconjugation of N-glycans to proteins in a host cell are also known in the art. Exemplary methods of generating a bifunctional binding protein provided herein are described in International Patent Application Publications WO2019/002512, WO2021/140143, WO2021/140144, and WO2022/053673. The biosynthetic pathways for N-glycosylation described in these publications can be used to synthesize the Man3 carrying bifunctional proteins described herein. In certain embodiments, the N-glycosylation is performed in host cells, resulting in production of Man3 carrying bifunctional proteins in the secretory pathway of the host cells. In certain embodiment, the host cell can be a Leishmania host cell. The Man3 glycosylated bifunctional proteins are further purified from the cell culture using any of the standard purification techniques known in the art.

Other methods to generate the bifunctional proteins provided herein can be used. For example, chemical conjugation or chemo-enzymatic modifications can be used to generate the bifunctional proteins provided herein.

It is understood that modifications which do not substantially affect the activity of the various embodiments of this invention are also provided within the definition of the invention provided herein. Accordingly, the following examples are intended to illustrate but not limit the present invention.

### 7. EXAMPLE

### 7.1 Example 1: Man3 Glycosylated Antibody Leads to Efficient Internalization and Lysosomal Compartment Targeting

PBMCs from 5 healthy donors were harvested from buffy coats using standard ficoll gradient method. Monocytes were isolated from PBMCs using CD14 Microbeads (Miltenyi ref. 130-050-201) following manufacturer's instructions. Monocytes were cultured in RPMI culture medium (Gibco, ref. 21875-034) supplemented with 10% heat inactivated fetal bovine serum, 1 mM sodium pyruvate (Sigma, ref. S8636), MEM Non-essential Amino Acid final concentration 1× (Sigma, ref 11140-03), 50 ng/ml recombinant human GM-CSF (R&D Systems, ref. 215-GM) and 20 ng/ml recombinant human IL-4 (R&D Systems, ref. 204-IL) for 6 days. Differentiation of monocytes into immature dendritic cells at day 6 was assessed by flow cytometry by staining for the following markers: CD14, CD1a, CD83, HLA-DR. Immature dendritic cells were confirmed to display the following phenotype: CD14−/low CDla+HLA−DR+CD83−/low.

Antibodies were prepared as follows. The monoclonal anti-TNF alpha antibodies, HUMIRA® (AbbVie) or Leishmania tarentolae CGP (CustomGlycan Platform) derived adalimumab variants (A-S, A-M), or Mabthera were re-buffered to 30 mM MES buffer pH 6.5 using ZebaSpin columns (ThermoFischer, US). The galactosylation and alpha 2,6-sialylation (to generate H-S and A-S variants) was performed using in vitro glycosylation (IVGE, Roche Diagnostics) in an 1-pot reaction at 37° C. under mild rotation according to the application note. The glycosylated mAb was purified from the reaction mixture with ProteinA sepharose (MabSelectSuRe or HiTrap MabSelect PrismA column GE Healthcare) according to manufacturer's recommendation using FPLC (Bio-Rad NGC, Germany). Thereafter, a desalting procedure using PD-10 (Sephadex 25, Sigma, Switzerland) was carried out for a buffer exchange to PBS pH 6 (137 mM NaCl, 2.7 mM KCl, 8.6 mM NaH2PO4, 1.4 mM Na2HPO4, Sigma, Switzerland) followed by sterile filtration using 0.2 μm PES filter (ThermoFisher, US). Quality and glycosylation of antibodies is shown in Table 1. N-glycans were analyzed using GlycoWorks RapiFluor-MS N-Glycan Kit (Waters, US) according to the manufacturer's instructions or by procainamide (PC) labelling of PNGaseF released glycans. For additional N-glycan analysis, the monoclonal antibody was either cleaved with IdeZ to F(ab′)2 and Fc/2 (For IgG1); or alternatively reduced to heavy and light chains (for IgG4), separated on SDS PAGE and bands were excised and enzymatic release of N-glycans from the monoclonal antibody was performed using PNGase F. Following release, glycans were labeled with procainamide (PC). PC-labeled N-glycans were analyzed by HILIC-UPLC-MS with fluorescence detection coupled to a mass spectrometer. Glycans were separated using an Acquity BEH Amide column. Data processing and analysis was performed using Unifi. Glucose units were assigned on the retention times of a procainamide-labeled dextran ladder. Glycan structures were assigned based on their m/z values and their retention times. Glycan forms and relative percentages were calculated based on peak areas. SE-HPLC analysis was performed at a concentration of 1 μg/μL on a MabPac (ThermoFischer, US) column and run according to the manufacturer's instructions and endotoxin levels were below 0.2 EU/mg (Endosafe®). Antibodies were labeled with pHrodo dye (pHrodo iFL Red STP Ester [amine-reactive], ThermoFisher, ref. P36011) according to manufacturer instructions. Table 1 shows the main N-glycan structure displayed by the antibodies used.

TABLE 1
Main glycoforms of pHrodo-labeled antibodies tested

| Antibody | Abbreviation | Glycan Nomenclature | Main N-glycan structure |
|---|---|---|---|
| Mabthera (rituximab) | MbT | A2F | 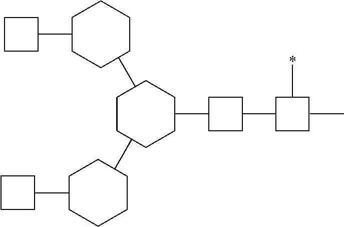 |
| HUMIRA ® (adalimumab) | H-M | A2F | 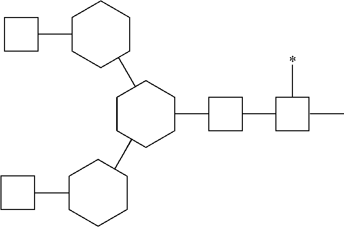 |
| Man3-adalimumab | A-M | Man3 (M3) | 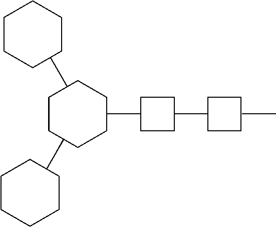 |
| Sialylated- adalimumab | A-S | A2G2S2 | 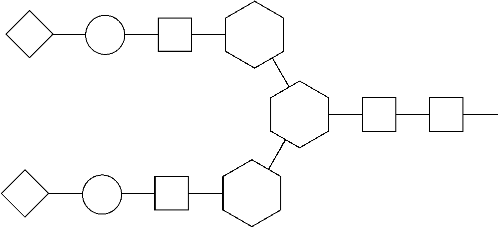 |
| Sialylated-HUMIRA ® | H-S | A2G2S2F | 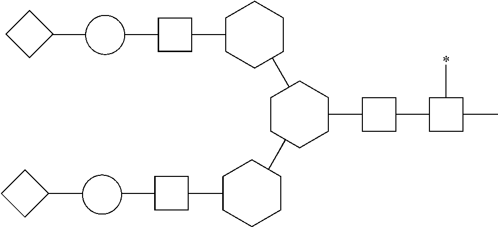 |

Table 1 shows the main N-glycoform (canonical N-297 position) displayed by indicated antibodies. The short nomenclature is typically used with IgG
associated naming system. It indicates the presence of core fucose, the number of galactoses and the presence of biantennary glycans. It is limited in
the number of structures and linkages in can describe but is often used for simplicity. Hexagon represents mannose (Man), white square is N-acetyl
glucosamine (GlcNAc), white circle is galactose (Gal), white diamond is sialic acid, N-acetyl neuraminic acid (Neu5Ac), and asterisk is fucose (Fuc).

Immature dendritic cells were incubated with pHrodo-labeled antibodies added at 10 g/ml for 6 hours. In some conditions, Fc receptor blocking reagent (Human TruStain FcX, biolegend ref 640922) was added according to manufacturer recommendation, 30 minutes before addition of pHrodo-labeled antibodies. After 6 hours of incubation with pHrodo-labeled antibodies, cells were acquired on a flow cytometer and pHrodo fluorescence intensity was recorded. pHrodo fluorescence is activated by acidic pH and therefore indicates that the internalized molecule has reach late endosomal and lysosomal compartments. pHrodo fluorescence intensity therefore correlates with the amount of molecule internalized and routed to the lysosomal pathway.

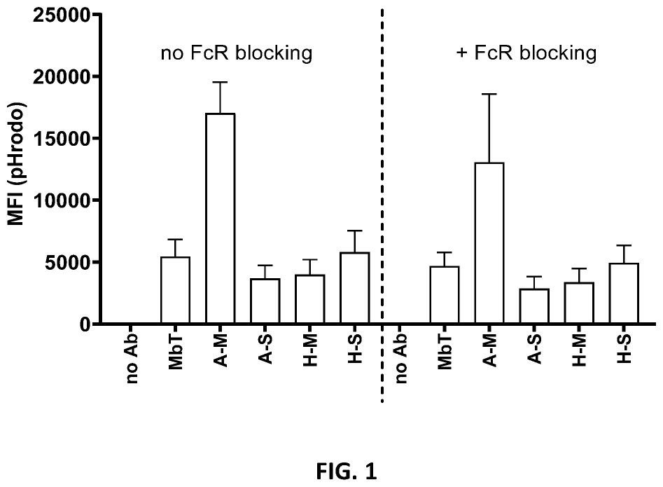

FIG. 1 shows that Man3 adalimumab (A-M) was significantly more internalized than adalimumab variants displaying different N-glycans. HUMIRA® (H-M) and sialylated HUMIRA® (H-S), as well sialylated (afucosylated) adalimumab (A-S) showed a similar low-level internalization as compared to Mabthera (MbT) an anti-CD20 antibody used as reference. Mabthera is displaying the same main N-glycan structure (A2F) as HUMIRA®. This indicates that Man3 glycan displayed by an antibody leads to internalization and targeting of the internalized antibody to lysosomal degradation. This internalization was not dependent on afucosylation since the afucosylated but sialylated adalimumab (A-S) was not more internalized than HUMIRA® or Mabthera. This internalization of Man3-antibody and is independent from Fc receptors since blocking these receptors did not decrease the internalization of A-M antibody. These data indicate that a specific receptor binding the Man3 structure is expressed by dendritic cells and mediates efficient endocytosis and routing to the lysosomal degradation pathway.

### 7.2 Example 2: Man3 Glycosylated Fab Antibody Leads to Efficient Internalization and Lysosomal Targeting on Human Macrophages

Antibodies were labeled with pHrodo dye (pHrodo iFL Red STP Ester [amine-reactive], ThermoFisher, ref. P36011) according to manufacturer instructions. The fluorescence of pHrodo is activated at low pH and therefore will allow for the visualization of protein internalization and targeting to the lysosomal pathway.

The pHrodo Degree of Labeling (DOL) for each antibody was determined as follows. Antibodies were diluted 1:2 in denaturing buffer and analyzed with Nanodrop at 280 nm and 560 nm wavelength (A280 and A560). Protein concentration and pHrodo DOL were calculated as follows.

$$Protein conc . [ M ] = \frac{protein conc . [ \frac{mg}{ml} ]}{M W [ g / mol ]} = \frac{protein conc . [ \frac{mg}{ml} ]}{144000}$$

$$DOL = \frac{{λ}_{max} * dilution factor}{{ε}_{dye} [ M - 1 cm - 1 ] * protein conc . [ M ]} = \frac{A 560 * 2}{65000 * protein conc . [ M ]}$$

MW is the molecular weight of the antibody used: 144000 g/Mol. λmax is the absorbance measured at 560 nm. εdye is the Extinction coefficient: 65000 M⁻¹ cm⁻¹. Dilution factor is 2.

Antibodies used in this experiment are shown in Table 2 along with their main N-glycan structure on Fc or Fab parts. Peripheral blood mononuclear cells (PBMCs) from human blood donors were harvested from buffy coats using standard ficoll gradient method. Monocytes were isolated from PBMCs using EasySep human monocytes isolation kit (StemCell ref. 19359). Monocytes were seeded in a tissue culture dish (Falcon, ref. 353003) at 5×10⁵ cells/ml in macrophage differentiation medium. Macrophage differentiation medium was: RPMI-1640 with L-glutamine (Sigma, R8758) supplemented with 10% fetal bovine serum (FBS, PanBiotech, P305500), 1% Pen/Strep (Sigma, P4333), 50 ng/ml hM-CSF (Peprotech, 300-25). Cells were incubated in a cell culture incubator at 37° C., 5% CO₂. Half of the cell culture medium was changed at day 3, by replacing with fresh macrophage differentiation medium. After a total of 6-7 days of differentiation, macrophages were harvested. Cell culture plates were rinsed with PBS 1× (Sigma, D8537) and incubated on ice in PBS 1×+1 mM EDTA (Invitrogen, 15575-020) for 15 min Macrophages were harvested by cell scraping and pipetting.

TABLE 2
Main glycoforms expressed by pHrodo labeled antibodies tested

| | | Engineered | | |
|---|---|---|---|---|
| | | Glycosites | Main N-glycan structure on | |
| Antibody | Abbreviation | (Eu numbering) | Fc | Main N-glycan structure on Fab |
| Mabthera (rituximab) | M-A2F | None | 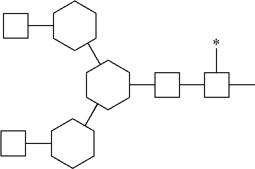 | No |
| HUMIRA ® (adalimumab) | H-A2F | None | 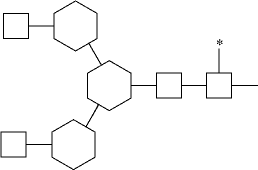 | No |
| Deglycosylate | H-PNGase | None | No | No |
| Humira | | | | |
| Fc-Man3- adalimumab | A-M3 | None | 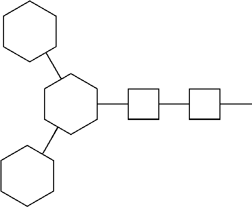 | No |
| Fab-Man3- adalimumab | A8486-M3 | LC: D86N HC: K84N | 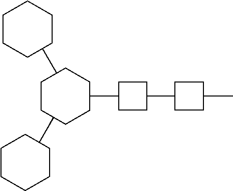 | 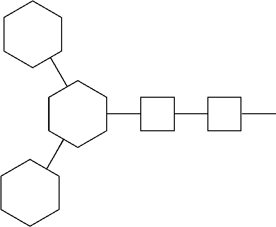 |
| Fab-Sia- adalimumab | A8486- A2G2S2 | LC: D86N HC: K84N | 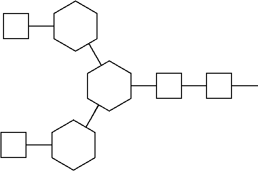 | 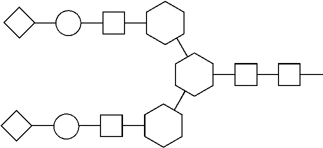 |

Table 2: Main N-glycan structure on Fc is on the canonical N-297 position. Main N-glycan structure on Fab means on the engineered glycosite by point
mutations (Eu numbering). LC is light chain, HC is heavy chain. Hexagon represents mannose (Man), white square is N-acetyl glucosamine (GlcNAc),
white circle is galactose (Gal), white diamond is sialic acid, N-acetyl neuraminic acid (Neu5Ac), and asterisk is fucose (Fuc).

Macrophage phenotype was verified by flow cytometry, staining for CD1a, CD14, CD206 and CD163. At least 90% of the cells were CD14+CD206+CD163+, confirming proper macrophage differentiation.

Macrophages were resuspended in internalization medium and seeded in 96-well flat bottom cell culture plates at 10⁵ cells/well in a total volume of 0.1 ml. Internalization medium is RPMI-1640 with L-glutamine supplemented with 2% FBS. Macrophages were preincubated with pooled human immunoglobulin (IVIg, Hizentra obtained from pharmacy) at 2 mg/ml final concentration for 30 min at 37° C. before pHrodo-labeled antibodies were added at 1l g/ml final concentration. Macrophages were incubated with pHrodo-antibodies+IVIg for 3 hours at 37° C. Cell cultures were then washed, macrophages were harvested using trypsinization and immediately acquired on a flow cytometer.

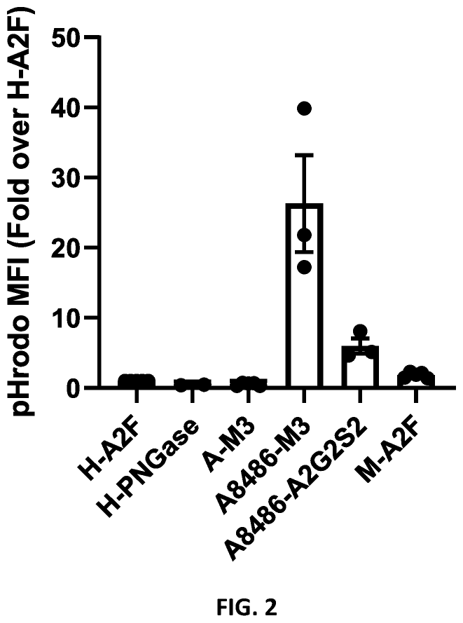

Mean fluorescence intensity (MFI) of pHrodo for gated single cell population was analyzed using standard flow cytometry software. MFI values were adjusted to pHrodo DOL. Adjusted MFI values were then normalized to Humira® (adalimumab) (H-A2F), for each donor. FIG. 2 shows the internalization data on macrophages. Mabthera (rituximab) (M-A2F) and Humira® (H-A2F) internalization was similar. Adalimumab variant displaying M3 glycans on the Fc part (A-M3) also internalized similarly to Humira® and Mabthera. Deglycosylation of Humira® and A-M3 (PNGase) led to a reduction of internalization of about 60%. Deglycosylation disrupts the Fc gamma receptor interaction as well as N-glycan receptor interaction and therefore represents the baseline internalization level. Humira® and Mabthera were internalized by Fc gamma receptors on macrophages. In contrast, the A8486-M3 variant, displaying M3 glycan on the Fab portion, were internalized by macrophages at a high level for all donors. Internalization of A8486-M3 was 17 to 39 fold higher than Humira® baseline depending on the donor. The A8486-A2G2S2 variant, which had 70% of sialic acid terminated N glycans on the Fab portion, was also internalized 6 fold more efficiently than Humira® baseline.

These results indicate that M3 glycans displayed on the Fab portion of an antibody lead to potent internalization and targeting to the lysosomal pathway by human macrophages. Man3-mediated lysosomal targeting was more potent than sialic acid-mediated lysosomal targeting. These data also show that the position of the Man3 glycan enables efficient internalization. Fab Man3 glycan leads to efficient internalization by human macrophages.

### 7.3 Example 3: Man3 Glycosylated Fab Antibody Leads to Efficient Internalization and Lysosomal Targeting in Human Dendritic Cells

Peripheral blood mononuclear cells (PBMCs) from 2 human blood donors were harvested from buffy coats using standard ficoll gradient method. Monocytes were isolated from PBMCs using EasySep Human Monocyte Isolation kit (Stemcell ref 19359C), following manufacturer's instructions.

Purified monocytes were resuspended in dendritic cell differentiation medium and seeded in T75 cell culture flasks at 3×10⁵ cells/cm². Dendritic cell differentiation medium included RPMI-1640 with L-glutamine (Sigma, R8758) supplemented with 10% fetal bovine serum (FBS, PanBiotech, P305500), 1% Pen/Strep (Sigma, P4333), 50 ng/ml (500 U/ml) rhGM-CSF (Peprotech, 300-03-20UG), 50 ng/ml rhIL-4 (Peprotech, 200-04-20UG). Cells were incubated for 3 days at 37° C., 5% CO₂. Half of the culture medium was replaced with fresh dendritic cell differentiation medium at day 3. At day 5, the medium was replaced by fresh dendritic cell differentiation medium supplemented with 50 ng/ml TNFα (Peprotech, AF-300-01A) and 10 ng/ml of IL-10 (Peprotech, 200-01B-10UG) and IL-6 (Peprotech, 200-06-5UG). The cells were further cultured for 2 additional days (total differentiation time 7 days). Cell cultures were washed and incubated on ice for 30 min in PBS 0.02% EDTA. Adherent cells were harvested by pipetting and washing with PBS.

Dendritic cell differentiation was verified by flow cytometry staining for CD1a, CD14 CD206 and CD163 using the following antibodies: Alexa Fluor647 anti-human CD163 (Biolegend 333619, 1:200), PerCP/Cyanine5.5 anti-human CD1a (Biolegend 300129, 1:200), PE anti-human CD14 (Biolegend 301805, 1:400), Alexa Fluor488 anti-human CD206 (Biolegend 321113, 1:200). Both donors showed at least 80% of differentiated dendritic cells as assessed by CD1a+ and CD206+.

Antibodies labeled with pHrodo were prepared as described in Example II. The antibodies and their glycan structure and pHrodo DOL are described in Table 2.

Dendritic cells were seeded in flat bottom 96-well plates at 0.5×10⁶ cells/well. Dendritic cells were preincubated with IVIg (Hizentra, obtained from pharmacy) at 1 mg/ml for 30 min at 37° C. before pHrodo labeled antibodies were added at final concentration of 10 μg/ml. Dendritic cells were incubated with antibodies for 6 hours. Cells were then harvested and acquired on a flow cytometer.

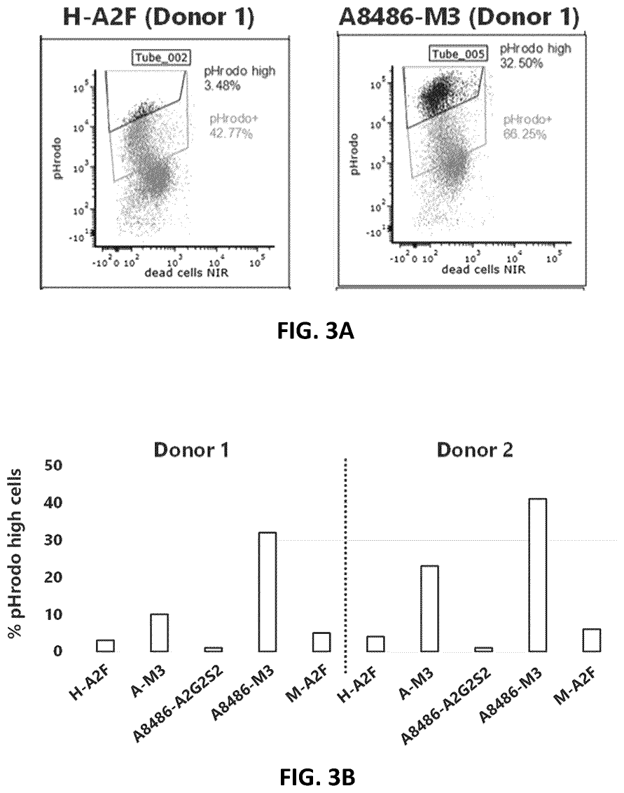

Flow cytometry data was analyzed using standard flow cytometry software. The percentage of cells which have performed efficient internalization was defined by gating the cells with high level of pHrodo internalization (pHrodo high population) as shown in FIG. 3A. FIG. 3B shows the percentage of pHrodo high cells for the different glycovariants tested. The data show that while Humira® (H-A2F) and Mabthera (M-A2F) were internalized with low efficiency by Dendritic cells, the A8486-M3 variant was highly internalized. The A-M3 variant showed higher internalization than Humira® or Mabthera although clearly inferior to the A-8486-M3 variant. Interestingly the A8486-A2G2S2 variant showed low internalization.

Overall these data in Examples I to III, both on macrophages and dendritic cells, show that Man3 glycans lead to potent internalization and targeting to the lysosome compared to classical glycan structures displayed by standard IgG antibodies represented by Humira® and Mabthera (A2F structure). These data also show that select positioning of Man3 glycans can lead to particularly efficient internalization—Man3 glycans displayed on Fab fragment led to much more potent internalization than Man3 glycans on Fc fragment (on canonical N297). Finally these data highlight that different glycan structures lead to different cell type targeting and efficiency of internalization and degradation. Sialylated glycans are internalized by macrophages but not by dendritic cells. Thus, by adapting glycan structure, and its position, one can direct a protein to specific cell types and therefore modulate the potency of internalization and degradation, as well as the functional outcome of the protein degradation.

### 7.4 Example 4: Internalization and Lysosomal Targeting of Man3 Glycosylated Fab Antibody in Human Macrophages is Mediated by Mannose Receptor and is Modulated by the Load of Man3 Displayed

To assess whether the internalization of antibodies displaying Man3 glycan on Fab is modulated by the amount of Man3 displayed, experiments using glycovariants with 1, 2 or 3 glycosites inserted in the Fab fragment was performed. Antibodies were purified and labelled with pHrodo as described in Example I. The Table 3 shows the antibody characteristics that were included in the study. All antibodies showed aggregate levels below 5% as assessed by size exclusion HPLC method and higher than 94% purity as assessed by reducing polyacrylamide gel electrophoresis analysis and had an endotoxin level lower than 1 EU/mg (LAL assay). Glycan and protein analytic methods are described in Example I.

TABLE 3
antibody characteristics

| | Engineered Glycosites | Main N-glycan |
|---|---|---|
| Antibody | (Eu numbering) | structure on Fc |
| H-A2F | None | 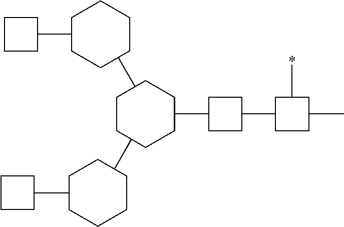 |
| A-8486-A2 | LC: D86N HC: K84N | 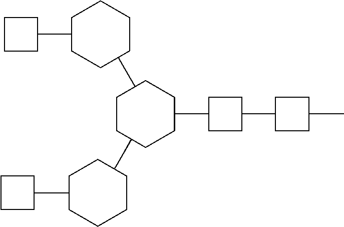 |
| A-84-M3 | HC: K84N | 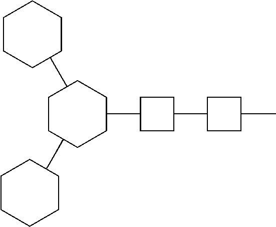 |
| A-8486-M3 | LC: D86N HC: K84N | 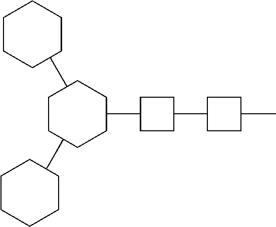 |
| A-8486- A2G2 | LC: D86N HC: K84N | 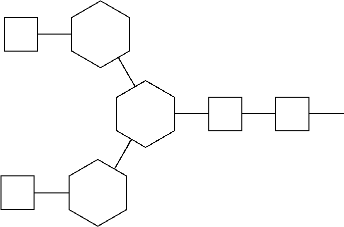 |
| A-8486- A2GalNAc2 | LC: D86N HC: K84N | 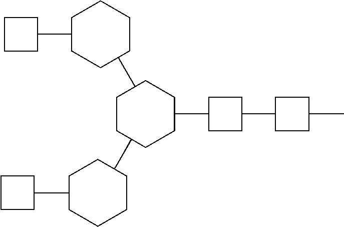 |
| 5C9-IgG4- A2F | None | 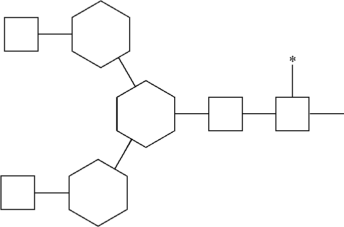 |
| | | 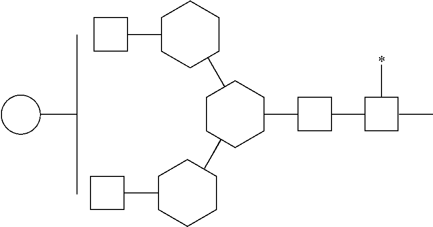 |
| 5C9- 84.86.162- M3 | LC: D86N HC: K84N HC: A162N | 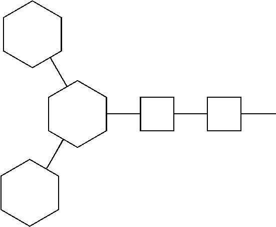 |

| | | | Endotoxin |
|---|---|---|---|
| | | | (EU/mg). |
| Antibody | Main N-glycan structure on engineered glycosites | N-Glycan | Assay LAL. |
| H-A2F | NA | 71% A2F | 0.1 |
| A-8486-A2 | 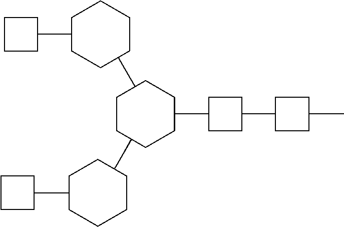 | Fab: 98% A2 Fc: 96% A2 | 0.9 |
| A-84-M3 | 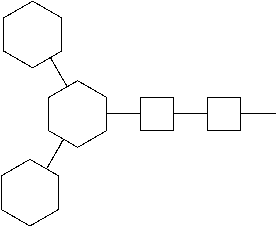 | 99% M3 | 0.1 |
| A-8486-M3 | 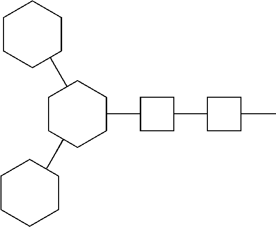 | 99% M3 | 0.2 |
| A-8486- A2G2 | 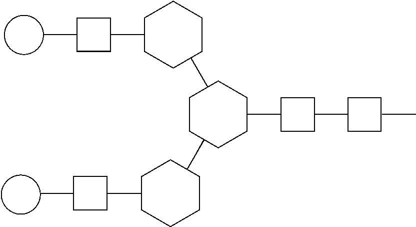 | Fab: 92% A2G2 Fc: 64% A2 | <0.5 |
| A-8486- A2GalNAc2 | 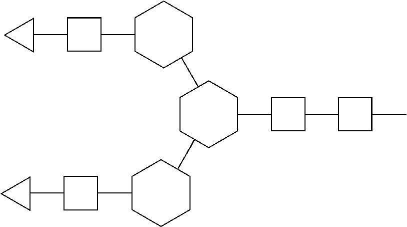 | Fab: 71% GalNAc2/ 15% GalNAc1 Fc: 95% A2 | <0.2 |
| 5C9-IgG4- | NA | 58.5% A2F | <1 |
| A2F | | 35.5% A2G1 | Eu/mg |
| 5C9- 84.86.162- M3 | 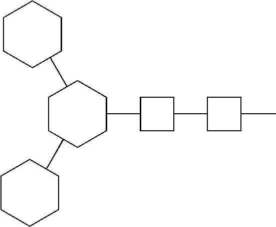 | Fab: 99% M3 Fc: 99% M3 | <1 EU/mg |

NA: not applicable. LC: light chain. HC: heavy chain. LAL: limulus amebocyte lysate. Hexagon represents mannose (Man), white square is N-acetyl
glucosamine (GlcNAc), triangle is N-acetyl galactosamine (GalNAc), white circle is galactose (Gal), and asterisk is fucose (Fuc). The vertical line means
that one Gal (open circle) can be at one or the other branch.

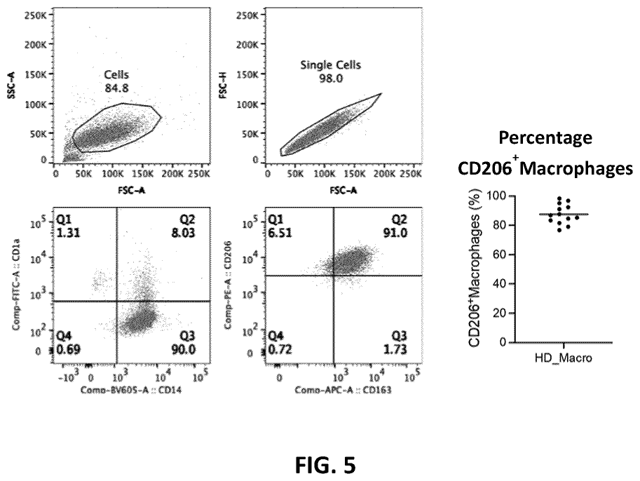

Human monocyte-derived M2 macrophages were produced as described in Example IL. Macrophage phenotype was verified by flow cytometry, staining for CD1a, CD14, CD206 and CD163. Typically, the macrophage purity was higher than 80%0 and the average percentage of CD206+ macrophages was higher than 8000 (FIG. 5).

Macrophages were resuspended in internalization medium and seeded in 96-well flat bottom cell culture plates at 10⁵ cells/well in a total volume of 0.1 ml. Internalization medium is RPMI-1640 with L-glutamine supplemented with 2% FBS. Macrophages were preincubated with pooled human immunoglobulin (IVIg, Hizentra obtained from pharmacy) at 2 mg/ml final concentration for 30 min at 37° C. before pHrodo-labeled antibodies were added at 1 μg/ml final concentration. Macrophages were incubated with pHrodo-antibodies+IVIg for 3 or 24 hours at 37° C. In some conditions, macrophages were incubated with mannan at 1 mg/ml for 30 min prior addition of pHrodo-labeled antibodies. Cell cultures were then washed, macrophages were harvested using trypsinization and immediately acquired on a flow cytometer. Mean fluorescence intensity (MFI) of pHrodo for gated single cell population was analyzed using standard flow cytometry software. MFI values were adjusted to pHrodo DOL to generate adjusted MFI values. Adjusted MFI of adalimumab variants (A-84 and A-84.86) were normalized to H-A2F reference (normalized MFI). The adjusted MFI of 5C9-84.86.162-M3 was normalized to 5C9-IgG4-A2F non-engineered reference antibody.

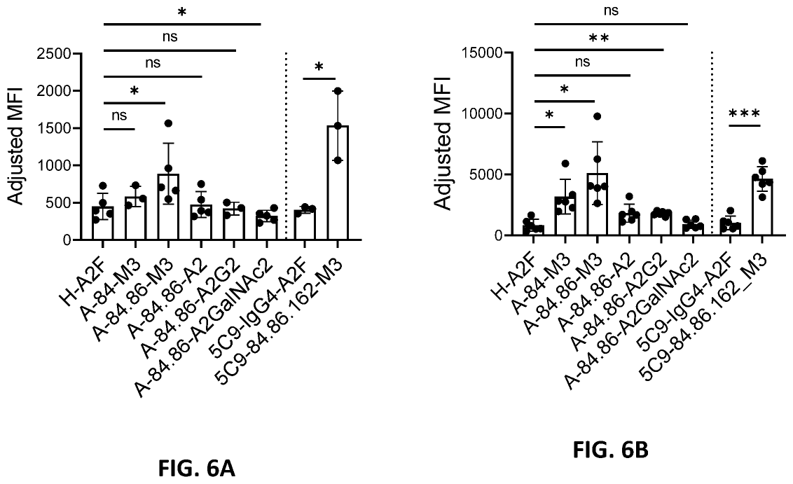

FIG. 6 and Table 4 show the adjusted MFI data. After 3 hours, A-84.86-M3 showed higher internalization than H-A2F reference. Similarly, 5C9-84.86.162-M3 showed higher internalization than 5C9-A2F reference. H-A2F and 5C9-A2F antibodies showed very similar internalization baseline showing that IgG1 (adalimumab) or IgG4 isotype (5C9) does not influence significantly the baseline of internalization by macrophages in these conditions. These data indicate that there is a trend for correlation between the load of M3 glycan displayed by antibodies and potency of internalization as the A-84-M3, with a single engineered glycosite per Fab, showed the lowest internalization while 5C9-84.86.162-M3 with 3 engineered glycosite per Fab, showed the highest internalization. These data and the data from example II indicate that the potency of internalization of A-8486-M3 by human-monocyte derived M2 macrophages ranged between 2 to more than 20 fold over H-A2F, indicating that variation of the potency occurs from donor to donor. At 3 hours, none of the non-M3 antibodies (A-84.86-A2G2, A-84.86-A2 and A-84.86-A2GalNAc2) showed more uptake than H-A2F (FIG. 6A), indicating that macrophages internalize quite selectively Man3-displaying antibodies. Importantly, the internalization of A-8486-M3 was reduced by addition of the Mannose Receptor competitive ligand Mannan at 1 mg/ml (Sigma, Ref. M7504), while uptake of H-A2F was not affected by mannan (Table 4). These data indicate that internalization of M3 antibodies by M2 macrophages is mediated by the Mannose Receptor (CD206). At 24 hours the level of internalization were higher, consistent with accumulation of pHrodo signal by cumulative cycles of internalization. Interestingly all M3 antibodies, including A-84-M3 showed higher internalization than the H-A2F and 5C9-IgG4-A2F baseline, indicating that even a single glycosite engineered M3 Fab antibody can be specifically recognized and internalized by M2 macrophages (FIG. 6B). A-8486-A2G2 antibody was also marginally (yet statistically significant) internalized as compared to H-A2F, at 24 hours.

TABLE 4
Mannan blocking experiment.

| | H-A2F | A-8486-M3 |
|---|---|---|
| Antibodies | 100 | 136.8 |
| Antibodies + Mannan | 94.9 | 66.2 |

Table 4 shows the mean normalized pHrodo MFI or indicated conditions from 2 macrophage donors. Macrophages were incubated 24 hours with pHrodo-labeled antibodies with or without Mannan.

### 7.5 Example 5: Man3 Glycosylated Fab Antibodies Lead to Potent In Vivo Depletion of a Blood Circulating Antigen

To assess the potency of antibodies produced in CGP and displaying Man3 terminated glycans (M3 structure) to deplete a circulating extracellular antigen, an experiment in rat was designed. Rats were injected with an antigen and with antibodies displaying M3 glycan structure on their Fab or control antibodies, specific for the antigen. The level of the circulating antigen in the serum of treated animals was quantified along time to measure the extend of depletion of the antigen from peripheral blood compartment. The Table 5 shows the antibody characteristics that were included in the study. All antibodies showed aggregate levels below 5% as assessed by size exclusion HPLC method and higher than 94% purity as assessed by reducing polyacrylamide gel electrophoresis analysis. All glycoengineered antibodies conserved high binding to the antigen, “HCA202”.

TABLE 5
Anitbody characteristics.

| | Engineered | | |
|---|---|---|---|
| | Glycosites | Main N-glycan | |
| | (Eu | structure on N297 | Main N-glycan structure |
| Antibody | numbering) | (Fc) | on engineered glycosites |
| H-A2F | None | 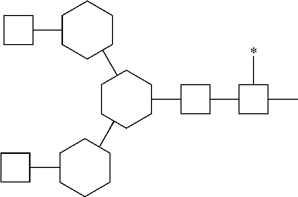 | NA |
| A-M3 | None | 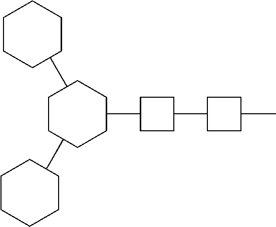 | NA |
| A-84-M3 | HC: K84N | 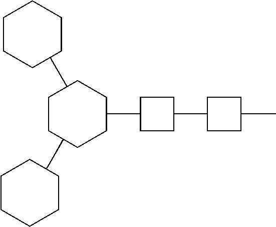 | 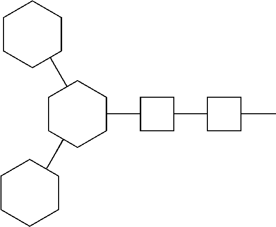 |
| A-8486- M3 | LC: D86N HC: K84N | 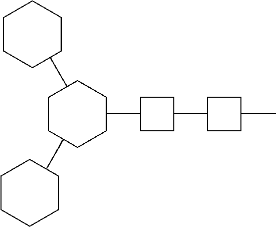 | 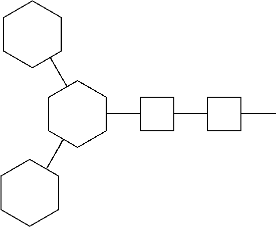 |
| A-8486- A2G2S2 | LC: D86N HC: K84N | 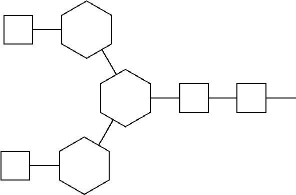 | 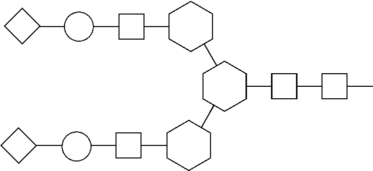 |

| | Binding to target | | Endotoxin |
|---|---|---|---|
| | antigen | | (EU/mg). |
| Antibody | (% of H-A2F antibody | N-Glycan | Assay LAL. |
| H-A2F | 100 | 71% | 0.1 |
| | | A2F | |
| A-M3 | 87 | 98% | 0.9 |
| | | M3 | |
| A-84-M3 | 66 | 99% | 0.1 |
| | | M3 | |
| A-8486-M3 | 85 | 99% | 0.2 |
| | | M3 | |
| A-8486-A2G2S2 | 108 | Fab: 48% S2/27% S1 | 0.2 |
| | | Fc: 52% A2/2% M3 | |

NA: not applicable. Binding to HCA202 was performed by ELISA and compared binding EC₅₀ values. LC: light chain. HC: heavy chain. Hexagon
represents mannose (Man), white square is N-acetyl glucosamine (GlcNAc), white circle is galactose (Gal), white diamond is sialic acid, N-acetyl
neuraminic acid (Neu5Ac), and asterisk is fucose (Fuc),

Wistar female rats (Janvier Labs, St Berthevin, France, ref. RjHan:WI) 180-220 g at start of experiment were injected i.v. bolus with anti-adalimumab Fab fragment HCA202 (Biorad, ref HCA202) (the antigen) at 0.5 mg/kg dose, 0.5 ml/rat. HCA202 compound was submitted prior to injection to an endotoxin removal step using Pierce™ High Capacity Endotoxin Removal Spin Columns (Thermofisher, ref 88274). Fifteen (15) min later rats were injected with antibodies (Table 4 and Table 5) or PBS. Blood samples were taken from jugular vein by puncture at following timepoints: 15 to 30 min after antibody injection, 1 hour, 6 hours and 24 hours. Terminal blood samples were collected at 48 hours from abdominal aorta. Blood samples were left for clotting 30 min at room temperature followed by centrifugation to collect serum.

Total HCA202 levels (antibody bound+free HCA202) were measured by ELISA method. Anti-Penta-His antibody (Qiagen, Ref. 34660) was coated on 96-well ELISA plates at 5 μg/ml in coating buffer (PBS pH 7.4, final composition: 8 mM Na-Phosphate; 8 mM K-Phosphate, 0.15 M NaCl, 10 mM KCl) overnight at 4° C. This antibody allows the capture of antibody bound and free HCA202 as HCA202 has an histidine tag at C terminus of heavy chain. Plates were washed 3 times with wash buffer (PBST=PBS with 0.05% v/v Tween-20). Blocking buffer (2% (w/v) Bovine serum albumin (BSA) in PBST) was added to each well and plates were incubated for 1-3 hours at room temperature. A 7-point calibration curve from 500 ng/ml to 0.7 ng/ml in 1:3 dilutions was made on separate dilution plates. For that, undiluted normal rat wistar serum was spiked with 5 μg/ml HCA202 and 3 fold molar excess of adalimumab (Humira). Spiked serum were incubated 10 min at room temperature to allow adalimumab/HCA202 immune complex formation. The spiked serum were diluted 10 fold (MRD10) by adding diluent B (2% (w/v) Bovine serum albumin (BSA) in PBST). A serial 1:3 dilution of the immune complex standard curve was performed using diluent B. Study samples were processed similarly. Study serum samples were diluted 10 fold in dilution plates (to achieve MRD10 samples) using diluent B. MRD10 samples were further diluted if needed in diluent A (1/10 wistar rat pooled serum diluted in 2% BSA+0.05% PBST) to achieve a signal within the linear range of the calibration standard curve. After blocking step, ELISA plates ware washed 3 times with wash buffer. Calibration standards and diluted study samples were added in duplicates to ELISA plates and incubated at room temperature for 1h. ELISA plates were washed 3 times with wash buffer and a Humira solution at 1000 ng/ml was added to each sample. ELISA plates were incubated for 1 h at room temperature. Plates were washed 3 times with wash buffer. A detection antibody solution was prepared by diluting goat anti-human kappa LC-HRP (Thermofisher, ref A18853) 1:5000 in diluent B. The detection antibody solution was added to the ELISA plates and incubated for 1h at room temperature, protected from light. ELISA plates were then washed 3 times with wash buffer and revealed by addition of TMB (3,3′,5,5′-tetramethylbenzidine) substrate followed by quenching with H₂SO₄. ELISA plates were read at 450 and 650 nM on a plate reader such as BioTek Synergy H1. Data analysis was made using standard software such as Gen5 (Biotek).

Antibody levels in serum sample can be quantified by ELISA method. The assay consists of a coating step with human TNFα to capture adalimumab and adalimumab variants present in the sample. Detection can be performed via an anti-human gamma HC specific HRP-tagged detection antibody. The assay therefore quantifies only free antibodies (having at least one Fab arm not bound to HCA202). Briefly, recombinant Human TNF-α (Peprotech, ref. AF-300-01A) is coated on 96-well ELISA plates, typically at 1 μg/ml in PBS pH 7.4 at 4° C. overnight. Blocking buffer, dilution buffer A and B and wash buffer are the same than used for the HC202 ELISA. Plates are washed 3 times and blocked with blocking buffer as described in the HCA202 ELISA. Typically, a 7-point calibration curve, for example from 333.3 ng/ml to 0.5 ng/ml in 1:3 dilutions is prepared by spiking pooled wistar rat serum diluted 10 fold (minimal required 10 fold dilution, MRD10) in dilution buffer B with 1 μg/ml adalimumab. Study serum samples are also diluted in dilution buffer B (MRD10 samples minimum). Diluted study samples and standard calibration curve samples are then transferred to the ELISA plate, after blocking step and incubated 1 h at room temperature. Plates are then washed 3 times and solution of detection antibody is added. Solution of detection antibody can be prepared by diluting for example a Goat Anti-Human IgG (γ-chain specific)-HRP (Sigma, ref. A6029) antibody (typical dilution 1:10′000) in dilution buffer B. ELISA plates are incubated with detection antibody typically 1h at room temperature, protected from light. Plates are then washed 3 times and revealed by adding TMB substrate as described in the HCA202 ELISA.

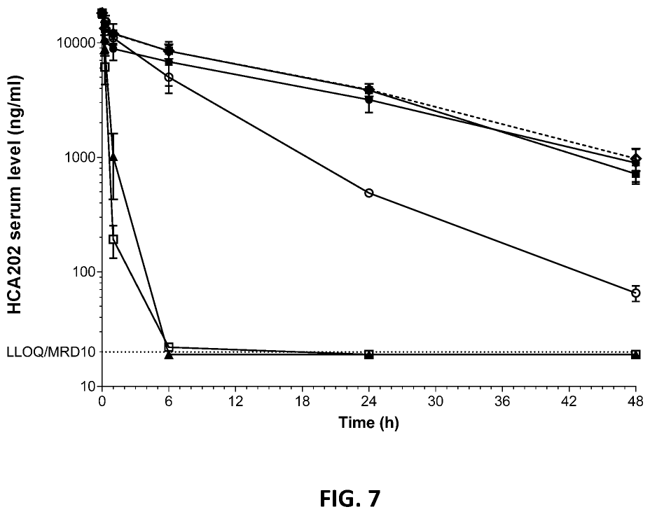

The FIG. 7 shows the data obtained for HCA202 (the antigen) levels. The Table 6 shows the HCA202 depletion data. When no antibody was injected (PBS condition), HCA202 decays slowly over the period of 48h, as expected for a Fab fragment. H-A2F (non-engineered adalimumab; Humira®) treatment led to increased levels of HCA202 at 24 and 48 hours timepoint. Treatment with A-M3 and A-8486-A2G2S2 also led to increased HCA levels as compared to PBS treatment (72% depletion from Czero with PBS vs 52-63% depletion with H-A2F, A-M3 and A-8486-A2G2S2 at 6h). Czero is the theoretical concentration (of HCA) in serum that would have been achieved immediately post injection, considering immediate homogeneous whole blood distribution. This shows that these antibodies have no depleting potency. In contrast injection of A-84-M3 and A-8486-M3, displaying exposed Man3 glycans, led to complete depletion of HCA202 as compared to non-depleting antibodies and PBS already at 1 hour (99 and 94% depletion), with non-detectable levels at 6 hours (Table 6).

TABLE 6
HCA202 depletion by Man3 glycosylated Fab antibodies

| | | | | A-8486- | | |
|---|---|---|---|---|---|---|
| | PBS | H-A2F | A-M3 | A2G2S2 | A-84-M3 | A-8486-M3 |
| % depletion at 1 h | 39 | 51 | 34 | 31 | 99 | 94 |
| % depletion at 6 h | 72 | 63 | 53 | 52 | 100 | 100 |

The table shows the % of HCA202 depletion from Czero.

These data highlight that antibodies displaying M3 glycans on their Fab fragment have a strikingly high potency to eliminate a circulating antigen from blood circulation in a very short time. In contrast M3 glycan displayed in the Fc fragment does not lead to active depletion.

### 7.6 Example 6: Man3 Glycosylated Fab Antibody is Targeted to the Liver In Vivo

To study the in vivo distribution of antibodies displaying Mannose terminated glycans (M3 structure), a study in mouse was designed with fluorescently-labeled antibodies displaying M3 or control glycans and using in vivo and ex-vivo tomography imaging.

Antibodies were labeled with CF750 labeling kit (Biotium, ref. 92221) for a volume of at least 1 mL at 1 mg/mL, following manufacturer's instructions. After labeling, the degree of labeling (DOL) was measured. The DOL ranged from 2.7 to 5.2 so antibodies were considered to be similarly labeled. The Table 5 in Example V shows the antibody characteristics that were included in the study. SKH1 immunocompetent hairless mice (Charles River Laboratories, ref. Crl:SKH1-hr) 5-6 weeks at experiment start were injected (intravenous bolus) with the CF750 labeled antibodies at 5 mg/kg dose. Mice were imaged using the FMT 2500TM fluorescence tomography in vivo imaging in the system (PerkinElmer), which collected both 2D surface fluorescence reflectance images (FRI) as well as 3D fluorescence tomographic (FMT) imaging datasets. For each animal 2 scans in decubitus dorsal position were performed (thorax area+abdomen area) with the FMT (NIR excitation laser 745 nm/emission 770-800 nm) at the following time points: 0.25 h, 1h, 3 h, 6h, 24 h and 48h. The collected fluorescence data were reconstructed by FMT 2500 system software (TrueQuant V2.0, PerkinElmer) for the quantification of the three-dimensional fluorescence signal in whole animal body. Three-dimensional regions of interest (ROI) were drawn encompassing the relevant biology on the thorax, abdomen and liver areas. The quantity and percentage of dose of labeled antibodies in each ROI was determined for each time point. At timepoint 6 hour and 48 hour, thyroids (with trachea), lungs, heart, liver, spleen, kidneys were harvested and submitted to FMT imaging.

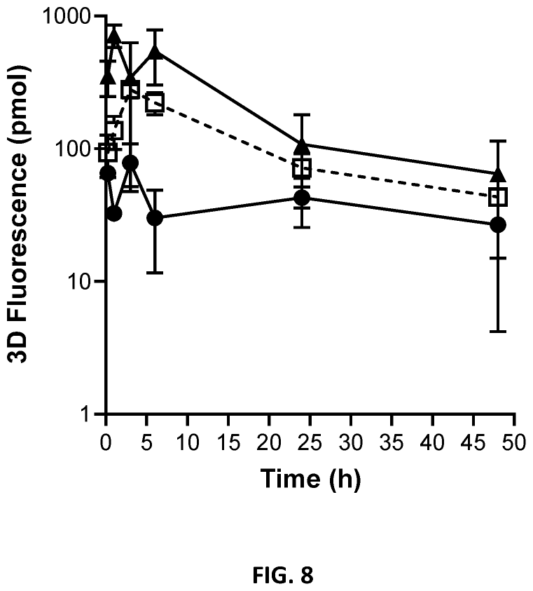

The FIG. 8 shows the FMT imaging data for thorax and liver ROI along time for each antibody. The Table 7 shows the FMT imaging data obtained on harvested organs at 6 hours. The non-engineered control antibody H-A2F (adalimumab, Humira®) showed a distribution profile with a low level of signal distributed liver area. Signal did not increase over time in the liver area (FIG. 8). At 6 hour timepoint, only 6% of injected dose of H-A2F was present in the liver (Table 7) while it was detected in all other organs. This distribution pattern is consistent with normal human IgG and characteristics of an antibody that is broadly distributed and still mainly present in the blood. The antibody A-M3 showed a distribution profile similar to H-A2F, with broad organ distribution. A-8486-M3 antibody showed a rapid and preferential distribution to the liver area (FIG. 8). At 6 hour timepoint 19% of the injected dose of A-8486-M3 was present in the liver (Table 7). A-8486-M3 antibody was absent from Thyroid, lungs, heart and kidneys and detectable only in the spleen. This pattern of distribution is characteristic of an antibody that is not present in the blood. This is consistent with the data showing that A-8486-M3 was internalized by Mannose Receptor on M2 macrophages (Example IV), showed a very led to a fast and potent depletion of a circulating antigen (Example V). These data support the assumption that antibodies displaying M3 structure on their Fab fragment are recognized by the Mannose Receptor (CD206), which triggers internalization and routing to the lysosomal degradation pathway.

TABLE 7
Organ distribution data at 6 hour.

| | H-A2F | A-M3 | A-8486-M3 |
|---|---|---|---|
| Liver | 6.1 | 16.4 | 18.7 |
| Lungs | 1.1 | 2.0 | 0.0 |
| Thyroid/Trachea | 0.5 | 0.4 | 0.0 |
| Heart | 0.7 | 0.8 | 0.0 |
| Kidneys | 2.0 | 2.3 | 0.0 |
| Spleen | 0.3 | 0.7 | 3.8 |

The table shows the average (N = 3) % of injected fluorophore dose in indicated harvested organs at 6 hour timepoint.

Throughout this application various publications have been referenced. The disclosures of these publications in their entireties are hereby incorporated by reference in this application in order to more fully describe the state of the art to which this invention pertains.

Although the invention has been described with reference to the examples provided above, it should be understood that various modifications can be made without departing from the spirit of the invention.

### REFERENCES

[1] O'Connor et al. “CCL2-CCR2 signaling in disease pathogenesis.” Endocrine, Metabolic & Immune Disorders-Drug Targets (Formerly Current Drug Targets-Immune, Endocrine & Metabolic Disorders) 15.2 (2015): 105-118.
[2] Gschwandtner et al. “More than just attractive: how CCL2 influences myeloid cell behavior beyond chemotaxis.” Frontiers in immunology 10 (2019): 2759.
[3] Liu et al. “Sox9 regulates self-renewal and tumorigenicity by promoting symmetrical cell division of cancer stem cells in hepatocellular carcinoma.” Hepatology 64.1 (2016): 117-129.
[4] Jo et al. Cross-talk between epidermal growth factor receptor and c-Met signal pathways in transformed cells. J Biol Chem 275 (2000): 8806-8011.
[5] Kim et al. “Targeting wnt signaling for gastrointestinal cancer therapy: Present and evolving views.” Cancers 12.12 (2020): 3638.
[6] Ciardiello et al. “Clinical development of therapies targeting TGFβ: current knowledge and future perspectives.” Annals of Oncology (2020).
[7] Zhang et al. “Tumor-associated macrophages: A promising target for a cancer immunotherapeutic strategy.” Pharmacological Research 161 (2020):105111.
[8] Cummings R D, Schnaar R L, Esko J D, Drickamer K, Taylor M E. Principles of Glycan Recognition. 2017. In: Varki A, Cummings R D, Esko J D, Stanley P, Hart G W, Aebi M, Darvill A G, Kinoshita T, Packer N H, Prestegard J H, Schnaar R L, Seeberger P H, editors. Essentials of Glycobiology [Internet]. 3rd ed. Cold Spring Harbor (NY): Cold Spring Harbor Laboratory Press; 2015-2017
[9] Drickamer K, Taylor ME. Recent insights into structures and functions of C-type lectins in the immune system. Curr Opin Struct Biol. 2015 October; 34:26-34. doi: 10.1016/j.sbi.2015.06.003. Epub 2015 Jul. 7. PMID: 26163333; PMCID: PMC4681411.
[10] Taylor M E, Drickamer K. Mammalian sugar-binding receptors: known functions and unexplored roles. FEBS J. 2019 May; 286(10):1800-1814. doi: 10.1111/febs.14759. Epub 2019 Feb. 6. PMID: 30657247; PMCID: PMC6563452.
[10] Taylor M E, Drickamer K. Structural insights into what glycan arrays tell us about how glycan-binding proteins interact with their ligands. Glycobiology. 2009 November; 19(11):1155-62. doi: 10.1093/glycob/cwp076. Epub 2009 Jun. 15. PMID: 19528664; PMCID: PMC2757572.
[11] Lepenies B, Lee J, Sonkaria S. Targeting C-type lectin receptors with multivalent carbohydrate ligands. Adv Drug Deliv Rev. 2013 August; 65(9):1271-81. doi: 10.1016/j.addr.2013.05.007. Epub 2013 May 30. PMID: 23727341.
[12] Allavena P, Chieppa M, Monti P, Piemonti L. From pattern recognition receptor to regulator of homeostasis: the double-faced macrophage mannose receptor. Crit Rev Immunol. 2004; 24(3):179-92. doi: 10.1615/critrevimmunol.v24.i3.20. PMID: 15482253.

1. A bifunctional binding protein comprising a first moiety that specifically binds to a target protein and a second moiety comprising a glycan comprising the structure: 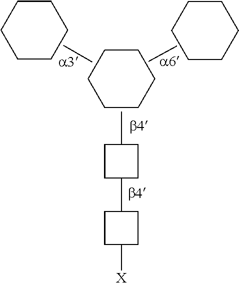
wherein the square represents an N-acetylglucosamine residue and the hexagon represents a mannose residue, and wherein X represents an amino acid residue of the bifunctional binding protein.

2. The bifunctional binding protein of claim 1, wherein the glycan further comprises a fucose residue at the N-acetylglucosamine that is directly attached to X.

3. The bifunctional binding protein of claim 1, wherein X is an asparagine residue in the bifunctional binding protein.

4. The bifunctional binding protein of claim 1, wherein the glycan consists of the structure of claim 1.

5. The bifunctional binding protein of claim 1, wherein the target protein is HER2, EGFR, HER3, VEGFR, CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, Frizzled receptors, Wnt, LRP5/6, CD38, CD73, TGF-β, Bombesin R, CAIX, CD13, CD44, v6, CXCR4, ErbB-2, Her2, Emmprin, Endoglin, EpCAM, EphA2, FAP-α, Folate R, GRP78, IGF-1R, Matriptase, Mesothelin, sMET/HGFR, MT1-MMP, MT6-MMP, Muc-1, PSCA, PSMA, Tn antigen, and uPAR, TSHRα, AChR-α1, noncollagen domain 1 of the α3 chain of type IV collagen (α3NC1), ADAMTS13, Desmoglein-1/3, or GPIb/IX, GPIIb/IIIa, GPIa/IIa, NMDA receptor, glutamic acid decarboxylase (GAD), amphiphysin and gangliosides GM1, GD3, GQ1B, MOG, SIRPα, CCR2, CSF-1R, LILRB1, LILRB2, VEGF-R, CXCR4, CCL2, CXCL12, CSF-1, CD47, or misfolded light chain and misfolded transthyretin.

6. The bifunctional binding protein of claim 1, wherein the second moiety specifically binds to:
a) a mannose 3 receptor, a Cluster of Differentiation 206 (CD206) receptor, a DC-SIGN (Cluster of Differentiation 209 or CD209) receptor, a C-Type Lectin Domain Family 4 Member G (LSECTin) receptor, a macrophage inducible Ca²⁺-dependent lectin receptor (Mincle); or
(b) any endocytic carbohydrate-binding receptor recognizing Man3GlcNAc2 structure.

7. (canceled)

8. (canceled)

9. (canceled)

10. The bifunctional binding protein of claim 1, wherein the first moiety comprises:
(a) a heavy chain variable region or a light chain variable region; or
(b) a Fab region of a monoclonal antibody.

11. (canceled)

12. The bifunctional binding protein of claim 1, wherein the bifunctional binding protein is an antibody.

13. The bifunctional binding protein of claim 12, wherein the antibody is (a) a monoclonal or polyclonal antibody; (b) recombinant; or (c) humanized, chimeric or fully human.

14. (canceled)

15. (canceled)

16. The bifunctional binding protein of claim 12, wherein the antibody has a glycan to protein ratio of 2 to 1, 4 to 1, 6 to 1, 8 to 1, or 10 to 1.

17. (canceled)

18. The bifunctional binding protein of claim 1, wherein the bifunctional binding protein is an autoantigen.

19. The bifunctional binding protein of claim 1, wherein at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% of the glycans of the bifunctional binding protein have the structure of the glycan of claim 1.

20. The bifunctional binding protein of claim 1, wherein the target protein is a cell surface molecule or a non-cell surface molecule.

21. The bifunctional binding protein of claim 20, wherein the cell surface molecule is a receptor or the non-cell surface molecule is an extracellular protein.

22. (canceled)

23. The bifunctional binding protein of claim 21, wherein the extracellular protein is an autoantibody, a hormone, a cytokine, a chemokine, a blood protein, or a central nervous system (CNS) protein.

24. The bifunctional binding protein of claim 20, wherein the target protein is capable of being bound by the first moiety.

25. A method of delivering a target protein to liver macrophages comprising: contacting the target protein with the bifunctional binding protein of claim 1 under conditions suitable to mediate endocytosis of the target protein.

26. A method of degrading a target protein comprising: contacting the target protein with the bifunctional binding protein of claim 1 under conditions suitable to mediate lysosomal degradation of the target protein by a host cell.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A pharmaceutical composition comprising the bifunctional binding protein of claim 1 and a pharmaceutically acceptable carrier.

40. A method of treating or preventing a disease in a patient comprising: administering to the patient the bifunctional binding protein of claim 1.

41. (canceled)

42. (canceled)

43. (canceled)

44. (canceled)

45. (canceled)

46. (canceled)

47. (canceled)

48. A kit comprising the bifunctional binding protein of claim 1 and instructions for administering the bifunctional molecule to an individual in need thereof.

49. (canceled)

50. A bifunctional binding protein, wherein the bifunctional binding protein (i) specifically binds to a target protein and (ii) comprises an N-glycan of the structure: 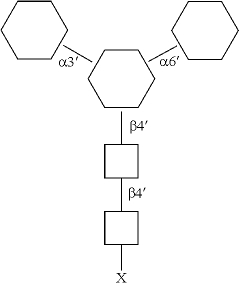
wherein the square represents an N-acetylglucosamine residue and the hexagon represents a mannose residue, and wherein X represents an amino acid residue of the bifunctional binding protein, wherein the N-glycan is linked to the bifunctional binding protein at 1, 2, 3, 4 or 5 N-glycosylation sites.

51. The bifunctional binding protein of claim 50, wherein the glycan further comprises a fucose residue at the N-acetylglusoamine that is directly attached to X and/or X is an asparagine residue in the bifunctional binding protein.

52. (canceled)

53. A population of bifunctional binding proteins according to claim 50, wherein for at least one of the N-glycosylation sites at a specified amino acid position of the bifunctional binding protein, at least 50%, at least 60%, at least 70%, at least 80%, at least 90% or 99% of N-glycosylation sites in the population are glycosylated with the N-glycan specified in claim 50.

54. The bifunctional binding protein of claim 50, wherein the N-glycosylation site(s) comprises one or more asparagine residues, wherein the asparagine residues are within a canonical consensus sequence N-X-S/T, N-X-C motifs, and non-canonical consensus motifs.

55. The bifunctional binding protein of claim 50, wherein the N-glycosylation sites) is introduced into the bifunctional protein by recombinant engineering.

56. (canceled)

57. The bifunctional binding protein of claim 50 wherein the glycan consists of the structure of claim 50.

58. (canceled)

59. (canceled)

60. (canceled)

61. (canceled)

62. The bifunctional binding protein of claim 50, wherein the bifunctional binding protein is an antibody.

63. (canceled)

64. (canceled)

65. (canceled)

66. (canceled)

67. (canceled)

68. The bifunctional binding protein of claim 62, wherein the antibody comprises an N-glycosylation site in the Fc domain of the antibody and wherein the N-glycan is linked to the N-glycosylation site in the Fc domain.

69. The bifunctional binding protein of claim 62, wherein the antibody comprises an N-glycosylation site in the heavy chain variable regions and/or light chain variable regions of the antibody and wherein the N-glycan is linked to the N-glycosylation site in the heavy chain variable regions and/or light chain variable regions.

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. A method of treating an acute condition associated with increased levels of a target protein, wherein the method comprises administering to a patient in need of treatment the bifunctional binding protein of claim 1, wherein the bifunctional binding protein (i) specifically binds to the target protein and (ii) comprises an N-glycan of the structure 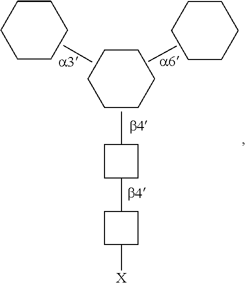
wherein the square represents an N-acetylglucosamine residue and the hexagon represents a mannose residue, and wherein X represents an amino acid residue of the bifunctional binding protein, wherein the N-glycan is linked to the bifunctional binding protein at a number of N-glycosylation sites that results in a half-life of the target protein of at most 5 minutes, 15 minutes, 30 minutes, 1 hour, 2 hours, 3 hours, 4 hours, 5 hours, or 6 hours in a patient after administration of the bifunctional binding protein to the patient, or that results in a half-life of the target protein of at most 1%, 5%, 10%, 15%, 20%, 25%, 30%, 35%, 40%, 45%, 50%, 60%, 70%, 75%, 80%, or at most 90% of the half-life of the target protein in the patient in the absence of any treatment.

104. (canceled)

105. (canceled)

106. (canceled)

107. A method of treating a chronic condition associated with increased levels of a target protein, wherein the method comprises administering to a patient in need of treatment the bifunctional binding protein of claim 1, wherein the bifunctional binding protein (i) specifically binds to the target protein and (ii) comprises an N-glycan of the structure 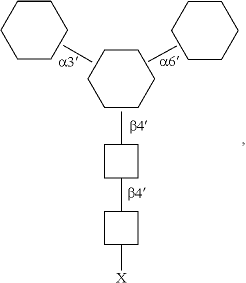
wherein the square represents an N-acetylglucosamine residue and the hexagon represents a mannose residue, and wherein X represents an amino acid residue of the bifunctional binding protein, wherein the N-glycan is linked to the bifunctional binding protein at a number of N-glycosylation sites that results in a half-life of the target protein of at least 1 day, 2 days, 3 days, or 4 days in the patient, or in a half-life of the target protein of at least 50%, 55%, 60%, 65%, 70%, 75%, 80%, 85%, 90%, or at least 95% of the half-life of the bifunctional binding protein without glycosylation in the patient.

108. (canceled)

109. (canceled)

110. (canceled)
